(19) United States
(12) Patent Application Publication
(10) Pub. No.: US 2023/0149387 A1
(43) Pub. Date: May 18, 2023

(54) TREATMENTS

(71) Applicant: Aerie Pharmaceuticals, Inc., Bedminster, NJ (US)

(72) Inventors: Mitchell A. deLong, Chapel Hill, NC (US); Kevin Stephen Carbajal, Raleigh, NC (US)

(21) Appl. No.: 17/917,853

(22) PCT Filed: Apr. 8, 2021
(86) PCT No.: PCT/US2021/026367

§ 371 Date: Oct. 7, 2022

Related U.S. Application Data
(60) Provisional application No. 63/007,103, filed on Apr. 8, 2020.

(51) Int. Cl. A61K 31/472; A61K 31/4439; A61K 33/22; A61K 47/18; A61K 47/26; A61K 47/10; A61K 31/519; A61K 47/02; A61K 9/00; A61P 31/14; A61P 27/02; A61P 11/00; A61K 31/4725

(52) U.S. Cl. CPC A61K 31/472; A61K 31/4439; A61K 33/22; A61K 47/183; A61K 47/26; A61K 47/186; A61K 47/10; A61K 31/519; A61K 47/02; A61K 9/008; A61K 9/0048; A61P 31/14; A61P 27/02; A61P 11/00; A61K 31/4725

(57) ABSTRACT

Described herein are compounds, combinations of compounds, compositions, formulations, and methods of treating viruses, viral load, and manifestations of viral infections.

### RELATED APPLICATIONS

This application is a national stage entry under 35 USC 371 of international patent application no. PCT/US2021/026367, filed Apr. 8, 2021, which claims priority of U.S. provisional patent application No. 63/007,103, filed Apr. 8, 2020, the entire content of each which is incorporated herein by reference.

### SUMMARY

Described herein are compounds, combinations of compounds, compositions, formulations, and methods of treating viruses, viral load, and manifestations of viral infections. In one embodiment, the herein described compounds, combinations of compounds, compositions, formulations, and methods are useful in treating coronaviruses.

Such compounds, combinations of compounds, compositions, formulations, and methods may be effective for treating viral conjunctivitis and/or pulmonary manifestations of viral infection. In some embodiments the viral infection is a coronavirus infection. In other embodiments, the infection results in severe acute respiratory syndrome coronavirus 2 (SARS-CoV-2). In one embodiment, the corona virus can lead to COVID-19.

It has been discovered that ROCK inhibitors, ROCK/JAK inhibitors and JAK inhibitors can mitigate a body's response to viral attack, including corona viruses and in some embodiments, COVID-19. The sites of attack of many viruses, including the corona viruses such as but not limited to CORVID-19, can include ocular tissues where they produce a viral conjunctivitis, and in the pulmonary system, where swelling, inflammation and fibrosis can occur.

In some embodiments, these virus manifestations can be treated using the herein described compounds, combinations of compounds, compositions, and formulations. In one embodiment, treatment can be with an eye drop formulation of these compounds, alone or in combination. In other embodiments, treatment can be with an inhaled formulation of these compounds, alone or in combination. In still other embodiments, the treatment can be systemic or local, or by another means as described herein.

In one embodiment, treatments as described herein can include the administration of one or more compound according to formula (1):

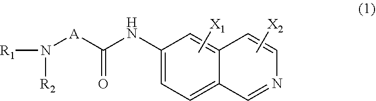

or a pharmaceutically acceptable salt thereof, wherein:

R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;

A is selected from the group consisting of —CH₂NH—, —CH(R₁₀)—, —C(CH₃)(R₁₀)—, —CH₂CH₂—CH(R₁₀)CH₂—, —CH₂CH₂CH(R₁₀)—, —CH₂CH(R₁₀)—, and —C(CH₃)(R₁₀)CH₂—;

each R₁₀ is independently selected from the group consisting of alkyl, alkenyl, alkynyl, amino, aryl, heteroaryl, cycloalkyl or heterocycloalkyl, any of which may be optionally substituted; and

X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl.

In some embodiments, a compound of Formula 1 can be administered with a ROCK/JAK inhibitor or a JAK inhibitor.

In another embodiment, treatments as described herein can include the administration of one or more compound according to of Formula (5):

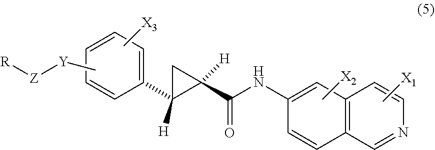

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;

Z is a direct bond or C₁₋₆ alkylene;

R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In some embodiments, a compound of Formula 5 can be administered with a ROCK/JAK inhibitor or a JAK inhibitor.

In another embodiment, treatments as described herein can include the administration of one or more JAK inhibitors including, but not limited to tofacitinib, decernotinib, INCB18424, baricitinib, CYT387, GLPG0634, AC-430, axitinib, ruxolitinib, fibotinib, and peficitinib.

In another embodiment, treatments as described can include a compound as described herein in combination with at least one antiviral compound.

In some embodiments, treatment may include combinations of any of the ROCK inhibitors and/or any of the JAK inhibitors described herein.

In another aspect, the disclosure may provide a method of treating an ocular disorder in a subject in need of treatment, comprising administering to the subject a compound or composition described herein. In some embodiments, the ocular disorder is viral conjunctivitis.

In another aspect, the disclosure may provide a method of improving the pulmonary function in a subject in need thereof, comprising topically administering to an pulmonary system of the subject a compound or composition described herein.

Other aspects and embodiments of the disclosure will become apparent in light of the following description.

### DETAILED DESCRIPTION

Compositions that include an isoquinoline ROCK inhibitors with an optional JAK inhibitor, are described. Also described herein are compounds, which include an isoquinoline compound that has both ROCK and JAK activity in the same molecule, and compounds which are compounds with mainly JAK inhibitory activity. Such compounds and compositions may be effective for treating viral conjunctivitis and/or pulmonary manifestations of viral infection. In some embodiments the viral infection is a coronavirus infection. In other embodiments, the infection results in severe acute respiratory syndrome coronavirus 2 (SARS-CoV-2) or COVID-19.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

“Acyl” or “carbonyl” refers to the group —C(O)R wherein R is selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, any of which may be optionally substituted, e.g., with one or more substituents. For example, when R is alkyl, such a group may be referred to as an alkylcarbonyl group.

“Administering” as used herein refers to administration of the compounds as needed to achieve a desired effect.

“Alkoxy” refers to the group —O—R wherein R is alkyl, alkenyl, alkynyl, cycloalkyl or heterocyclyl, any of which may be optionally substituted, e.g., with one or more substituents.

“Alkyl” refers to a saturated aliphatic hydrocarbon chain, which may be straight or branched. An alkyl group may have an indicated number of carbon atoms. For example, C₁-C₁₂ alkyl refers to an alkyl group having from 1 to 12 (inclusive) carbon atoms. C₁-C₄ alkyl refers to an alkyl group having 1, 2, 3 or 4 carbon atoms, such as methyl, ethyl, n-propyl, isopropyl, n-butyl, sec-butyl or tert-butyl. An alkyl group may be optionally substituted, e.g., with one or more substituents.

“Alkylene” refers to a divalent alkyl group, e.g., —CH₂—, —CH₂CH₂—, —CH₂CH₂CH₂— or —CH₂CH(CH₃)CH₂—. An alkyl or alkylene may be optionally substituted, e.g., with one or more substituents.

“Alkenyl” refers to a straight or branched hydrocarbon chain having one or more double bonds. An alkenyl group may have an indicated number of carbon atoms. For example, C₂-C₁₂ alkenyl refers to an alkenyl group having from 2 to 12 (inclusive) carbon atoms. C₂-C₄ alkenyl refers to an alkenyl group having 2, 3 or 4 carbon atoms. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkenylene” refers to a divalent alkenyl, e.g., —CH═CH—, —CH═CH₂CH₂— or —CH═C═CH—. An alkenyl or alkenylene may be optionally substituted, e.g., with one or more substituents.

The term “alkynyl” refers to a straight or branched hydrocarbon chain having one or more triple bonds. An alkynyl group may have an indicated number of carbon atoms. For example, C₂-C₁₂ alkynyl refers to an alkynyl group having from 2 to 12 (inclusive) carbon atoms. C₂-C₄ alkynyl refers to an alkynyl group having 2, 3 or 4 carbon atoms. Examples of alkynyl groups include, but are not limited to, ethynyl, propargyl, and 3-hexynyl. One of the triple bond carbons may optionally be the point of attachment of the alkynyl substituent. The term “alkynylene” refers to a divalent alkynyl, e.g., —C≡C— or —C≡C≡CH₂—. An alkynyl or alkynylene may be optionally substituted, e.g., with one or more substituents.

“Amino” refers to the group —NR′R″ wherein R′ and R″ are each independently selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, or R′ and R″, together with the nitrogen to which they are attached, may form a ring. Examples of amino groups include, but are not limited to, —NH₂, alkylamino groups such as —NHCH₃, —NHCH₂CH₃ and —NHCH(CH₃)₂, dialkylamino groups such as —N(CH₃)₂ and —N(CH₂CH₃)₂, and arylamino groups such as —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The groups R′ and R″ may be optionally substituted, e.g., with one or more substituents, or when R′ and R″ together with the nitrogen to which they are attached form a ring, the ring may be optionally substituted, e.g., with one or more substituents.

“Aryl” refers to an aromatic monocyclic, bicyclic, or tricyclic hydrocarbon ring system, wherein any ring atom capable of substitution can be substituted (e.g., with one or more substituents). The substituents may be positioned at various locations on an aryl group. For example, substituents on a phenyl group may be located at an ortho-position, a meta-position, the para-position, or combinations thereof. Examples of aryl groups include, but are not limited to, phenyl, naphthyl, and anthracenyl.

“Arylalkyl” refers to an alkyl group in which an alkyl hydrogen atom is replaced with an aryl group. Arylalkyl includes groups in which more than one hydrogen atom has been replaced with an aryl group. Examples of arylalkyl groups include but are not limited to benzyl, 2-phenylethyl, 3-phenylpropyl, 9-fluorenyl, benzhydryl, and trityl groups. Arylalkyl groups can be optionally substituted, e.g., with one or more substituents on either the alkyl portion or the aryl portion of the arylalkyl group.

“Aryloxy” refers to the group —O—R wherein R is aryl or heteroaryl, either of which may be optionally substituted, e.g., with one or more substituents.

“Buffer” or “buffer system” refers to a compound or combination of compounds that provide a buffering system in solution that exhibits buffering capacity, that is, the capacity to neutralize, within limits, either acids or bases with relatively little or no change in the original pH. The term “buffering capacity” is defined to mean the millimoles (mM) of strong acid or base (or respectively, hydrogen or hydroxide ions) required to change the pH by one unit when added to one liter (a standard unit) of the buffer solution. The buffer capacity will depend on the type and concentration of the buffer components.

“Carboxyl” refers to the group —C(═O)OR, wherein R is selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl any of which may be optionally substituted, e.g., with one or more substituents.

“Carbonylamino” or “amido” refers to the group —C(O)NR′R″ wherein R′ and R″ are independently selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, or R′ and R″, together with the nitrogen to which they are attached, may form a ring. The groups R′ and R″ may be optionally substituted, e.g., with one or more substituents, or when R′ and R″ together with the nitrogen to which they are attached form a ring, the ring may be optionally substituted, e.g., with one or more substituents.

“Cycloalkyl” refers to nonaromatic, saturated or partially unsaturated monocyclic, bicyclic, tricyclic or polycyclic hydrocarbon groups. Cycloalkyl groups may include about 3 to about 12 carbon atoms. For example, monocyclic cycloalkyl groups may include 3 to 10 carbon atoms, e.g., 3, 4, 5, 6, 7 or 8 carbon atoms. Bicyclic carbocyclic groups contain 8 to 12 carbon atoms, e.g., 9 or 10 carbon atoms. Any ring atom can be substituted (e.g., with one or more substituents). Cycloalkyl groups include fused, spiro, and bridged bicyclic ring systems. Examples of cycloalkyl groups include, but are not limited to, cyclopropyl, cyclobutyl, cyclopentyl, cyclohexyl, cyclohexenyl, cyclohexadienyl, methylcyclohexyl, adamantyl, norbornyl and norbornenyl.

“Cycloalkylalkyl”, as used herein, refers to an alkyl group substituted with a cycloalkyl group.

“Excipient” refers to physiologically compatible additives useful in preparation of a pharmaceutical composition. Examples of pharmaceutically acceptable carriers and excipients can, for example, be found in Remington Pharmaceutical Science, 16^{th} Ed.

“Haloalkyl” as used herein refers to an alkyl group in which one or more hydrogen atoms are replaced with a halogen, and includes alkyl moieties in which all hydrogens have been replaced with halogens (e.g., perfluoroalkyl such as CF₃).

“Halogen” or “halo” refers to fluoro, chloro, bromo or iodo moieties.

“Heteroalkyl” refers to an alkyl group, as defined herein, wherein at least one carbon atom of the alkyl group is replaced with a heteroatom. Suitable heteroalkyl groups include, but are not limited to, methoxymethyl (—CH₂—O—CH₃).

“Heteroaryl” or “heteroaromatic” refers to an aromatic monocyclic, bicyclic or tricyclic ring having one or more heteroatoms. For example a heteroaryl group may be an aromatic 5-8 membered monocyclic ring having 1-4 heteroatoms, an 8-12 membered bicyclic ring having 1-6 heteroatoms, or an 11-14 membered tricyclic ring system having 1-9 heteroatoms. Heteroaryl groups can contain fused rings, which are rings that share one or more common atoms. Any ring atom capable of substitution can be substituted (e.g., with one or more substituents). Examples of heteroaryl groups include, but are not limited to, tetrazoylyl, triazolyl, thienyl, thiazolyl, isothiazolyl, purinyl, pyrimidyl, pyridyl, pyrazinyl, pyridazinyl, pyrrolyl, imidazolyl, pyrazolyl, oxazolyl, isoxazolyl, furanyl, quinolinyl, isoquinolinyl, quinoxalinyl, quinazolinyl, cinnolinyl, indolyl, isoindolyl, indolizinyl, indazolyl, benzimidazolyl, phthalazinyl, pteridinyl, carbazolyl, carbolinyl, phenanthridinyl, acridinyl, phenanthrolinyl, phenazinyl and naphthyridinyl.

The term “heteroarylalkyl”, as used herein, refers to an alkyl group substituted with a heteroaryl group.

“Heteroatom” refers to an atom other than carbon in the ring of a heterocyclic group or a heteroaromatic group or the chain of a heteroalkyl group. For example, heteroatoms may be selected from the group consisting of nitrogen, oxygen, silicon, phosphorus and sulfur. Particularly suitable heteroatoms are nitrogen, oxygen and sulfur. Groups containing more than one heteroatom may contain different heteroatoms.

“Heterocyclyl” or “heterocycloalkyl” refers to a nonaromatic, saturated or partially unsaturated hydrocarbon ring system containing at least one heteroatom. Heterocyclyl groups may include about 3 to about 12 member atoms. For example, monocyclic cycloalkyl groups may include 3 to 10 member atoms, e.g., 3, 4, 5, 6, 7 or 8 member atoms. Bicyclic carbocyclic groups contain 8 to 12 member atoms, e.g., 9 or 10 member atoms. Any ring atom capable of substitution can be substituted (e.g., with one or more substituents). Heterocyclyl groups include fused, spiro, and bridged bicyclic ring systems. Examples of heterocyclyl groups include, but are not limited to, epoxy, tetrahydrofuranyl, homopiperidinyl, tetrahydrothienyl, tetrahydropyranyl, piperidinyl, piperazinyl, morpholinyl, pyrrolinyl, pyrimidinyl, pyrrolidinyl, indolinyl, tetrahydropyridinyl, dihydropyran, thianthrene, pyran, benzopyran, xanthene, phenoxathiin, phenothiazinyl, furazanyl, lactones, lactams such as azetidinones and pyrrolidinones, sultams, sultones, and the like.

The term “heterocyclylalkyl” or “heterocycloalkylalkyl”, as used herein, refers to an alkyl group substituted with a heterocyclyl group.

“Hydroxy” or “hydroxyl” refers to the group —OH.

“Linker” means a chain of n member atoms where n is an integer from 1 to 4.

“Member atom” means a carbon, nitrogen, oxygen or sulfur atom. Member atoms may be substituted up to their normal valence.

The term “mercapto” or “thiol” refers to an —SH radical. The term “thioalkoxy” or “thioether” refers to an —S-alkyl radical. The term “thioaryloxy” refers to an —S-aryl radical.

The term “ocular disorder” as used herein includes, but is not limited to, viral infections of the eye, viral conjunctivitis, coronaviral conjunctivitis, COVID-19 conjunctivitis, allergy, dry eye, and corneal wound healing. A “method of treating an ocular disorder” may refer to a method of treating viral infections of the eye, viral conjunctivitis, coronaviral conjunctivitis, COVID-19 conjunctivitis, allergy, dry eye, and corneal wound healing.

The term “oxo” refers to an oxygen atom, which forms a carbonyl when attached to carbon, an N-oxide when attached to nitrogen, and a sulfoxide or sulfone when attached to sulfur. The term “thioxo” refers to a sulfur atom, which forms a thiocarbonyl when attached to carbon.

“Phosphonate” refers to —P(O)(OR)₂, wherein each R is independently selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, each of which may be optionally substituted, e.g., with one or more substituents.

“Ring” means a collection of member atoms that are cyclic. Rings may be carbocyclic, aromatic, or heterocyclic or heteroaromatic, and may be substituted or unsubstituted, and may be saturated or unsaturated. Ring junctions with the main chain may be fused or spirocyclic. Rings may be monocyclic or bicyclic. Rings contain at least 3 member atoms and at most 12 member atoms. Monocyclic rings may contain 3 to 10 member atoms and bicyclic rings may contain from 8 to 12 member atoms. Bicyclic rings themselves may be fused or spirocyclic. Rings may be optionally substituted or unsubstituted, e.g., with one or more substituents.

“ROCK inhibitor”, or “ROCKi” as used herein refers to an inhibitor of a Rho-associated protein kinase (ROCK). Non-limiting examples of ROCK inhibitors include netarsudil, fasudil and ripasudil.

“JAK inhibitor”, or “JAKi” as used herein refers to an inhibitor of a Janus protein kinase (JAK), these include JAK1, JAK2, JAK3, and TYK2. Non-limiting examples of JAK inhibitors include tofacitinib, decernotinib, INCB18424, baricitinib, CYT387, GLPG0634, AC-430, axitinib, ruxolitinib, fibotinib, and peficitinib.

“Substituent” refers to a group “substituted” on a group such as an alkyl, alkylene, alkenyl, alkynyl, cycloalkyl, cycloalkylalkyl, heterocyclyl, heterocyclylalkyl, aryl, arylalkyl, heteroaryl or heteroarylalkyl group, at any substitutable atom of that group. Suitable substituents include, without limitation: acyl, alkoxy, alkyl, alkenyl, alkynyl, amino, aryl, arylalkyl, carbonylamino, carboxy, cycloalkyl, cycloalkylalkyl, cyano, halo, haloalkyl, heteroalkyl, heteroaryl, heteroarylalkyl, heterocyclyl, heterocyclylalkyl, hydroxy, nitro, oxo (e.g., C═O), phosphonate, sulfinyl, sulfonyl, sulfonate, sulfonamido, thioamido, thiol, thioalkyl, thioxo (e.g., C═S), and ureido. In embodiments, substituents on a group are independently any one single, or any combination of the aforementioned substituents. In embodiments, a substituent may itself be substituted with any one of the above substituents.

The above substituents may be abbreviated herein, for example, the abbreviations Me, Et, Ph, Bn and Ac represent methyl, ethyl, phenyl, benzyl and acetyl respectively. A more comprehensive list of the abbreviations used by organic chemists of ordinary skill in the art appears in the first issue of each volume of the Journal of Organic Chemistry; this list is typically presented in a table entitled Standard List of Abbreviations. The abbreviations contained in said list, and all abbreviations used by organic chemists of ordinary skill in the art, are hereby incorporated by reference.

“Sulfinyl” refers to a —S(═O)R group, wherein R is selected from the group consisting of alkyl, alkenyl, alkynyl, aryl, cycloalkyl, heterocyclyl, heteroaryl, arylalkyl, cycloalkylalkyl, heteroarylalkyl and heterocyclylalkyl, any of which may be optionally substituted (e.g., with one or more substituents).

“Sulfonic acid” and “sulfonate” refer to —S(O)₂OH and —S(O)₂O— groups respectively

“Sulfonyl” refers to a —S(O)₂R group, wherein R is selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, any of which may be optionally substituted (e.g., with one or more substituents).

“Sulfonamido” refers to a —S(O)₂NR′R″ group wherein R′ and R″ are independently selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, any of which may be optionally substituted (e.g., with one or more substituents).

“Therapeutically effective amount” refers to a dosage of the compounds or compositions effective for influencing, reducing or inhibiting the activity of or preventing activation of a kinase. This term as used herein may also refer to an amount effective at bringing about a desired in vivo effect in an animal, preferably, a human, such as reduction in intraocular pressure.

“Thioalkyl” refers to the group —S-alkyl.

“Thioamido” refers to —C(S)NR′R″ wherein R′ and R″ are independently selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, or R′ and R″, together with the nitrogen to which they are attached, may form a ring. The groups R′ and R″ may be optionally substituted, e.g., with one or more substituents, or when R′ and R″ together with the nitrogen to which they are attached form a ring, the ring may be optionally substituted, e.g., with one or more substituents.

“Treat” or “treating” as used herein refers to administering a regimen to the subject, e.g., the administration a compound or composition described herein, such that the disorder or at least one symptom of the disorder is healed, alleviated, relieved, altered, remedied, ameliorated, and/or improved. Treating includes administering an amount effective to alleviate, relieve, alter, remedy, ameliorate, improve and/or affect the disorder or the symptoms of the disorder. The treatment may inhibit deterioration or worsening of a symptom of a disorder.

“Ureido” refers to —N(R)C(O)NR′R″, wherein each R, R′ and R″ is independently selected from the group consisting selected from the group consisting of hydrogen, alkyl, alkenyl, alkynyl, aryl, cycloalkyl, heterocyclyl, heteroaryl, arylalkyl, cycloalkylalkyl, heteroarylalkyl and heterocyclylalkyl, any of which may be optionally substituted (e.g., with one or more substituents).

Where substituent groups are specified by their conventional chemical formulae, written from left to right, they optionally encompass substituents resulting from writing the structure from right to left, e.g., —CH₂NH— optionally also recites —NHCH₂—. While certain lists of substituent groups include a group shown in both orientations, it should be expressly understood that any substituent group written in a certain direction (e.g., left to right) also encompasses the same group in the other direction (e.g., right to left).

In accordance with a convention used in the art, the group:

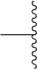

is used in structural formulas herein to depict a bond that is the point of attachment of the moiety or substituent to the core or backbone structure.

For compounds described herein, groups and substituents thereof are to be selected in accordance with permitted valence of the atoms and the substituents, such that the selections and substitutions result in a stable compound, e.g., which does not spontaneously undergo transformation such as by rearrangement, cyclization, elimination, etc.

It specifically is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

All percentages, ratios, and proportions used herein are percent by weight per volume (% wt/vol or w/v) unless otherwise specified.

### Compounds

Compounds that may be used in compositions described herein include isoquinoline compounds. Such compounds and the compositions including them may have kinase inhibitory activity and thus may be useful in influencing or inhibiting the action of kinases, and in treatment and/or prevention of diseases or conditions influenced by kinases. Exemplary kinases that may be influenced include, but are not limited to, ROCK-I, ROCK-II, PKA, PKC, CAM Kinases, GRK-2, GRK-3, GRK-5 or GRK-6. For example, the kinase inhibited may be a Rho-associated protein kinase (ROCK).

Compounds that may be used in compositions and methods described herein include compounds of Formula (1):

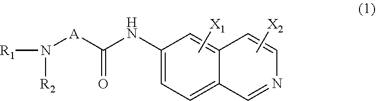

or a pharmaceutically acceptable salt thereof, wherein:

R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;

A is selected from the group consisting of —CH₂NH—, —CH(R₁₀)—, —C(CH₃)(R₁₀)—, —CH₂CH₂—CH(R₁₀)CH₂—, —CH₂CH₂CH(R₁₀)—, —CH₂CH(R₁₀)—, and —C(CH₃)(R₁₀)CH₂—;

each R₁₀ is independently selected from the group consisting of alkyl, alkenyl, alkynyl, amino, aryl, heteroaryl, cycloalkyl or heterocycloalkyl, any of which may be optionally substituted; and

X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl.

In some embodiments of Formula (1), X₁ is hydrogen, X₂ is hydroxy, R₁ is alkyl (e.g., methyl), R₂ is alkyl (e.g., methyl), A is —CH(R₁₀)—, and R₁₀ is aryl (e.g., phenyl).

In some embodiments of Formula (1), X₁ is hydrogen, X₂ is hydroxy, R₁ and R₂ together form a heterocyclyl ring, A is —CH(R₁₀)—, and R₁₀ is alkyl.

In some embodiments of Formula (1), X₁ and X₂ are hydrogen, R₁ is alkyl (e.g., methyl), and R₂ is alkyl (e.g., methyl), A is —CH(R₁₀)—, and R₁₀ is heteroaryl (e.g., thienyl).

In some embodiments of Formula (1), X₁ and X₂ are hydrogen, R₁ is hydrogen, and R₂ is hydrogen, A is —CH₂CH(R₁₀)—, and R₁₀ is a substituted aryl group.

In some embodiments of Formula (1), X₁ is hydrogen, X₂ is hydroxy, R₁ is alkyl (e.g., methyl), R₂ is alkyl (e.g., methyl), A is —CH(R₁₀)—, and R₁₀ is heteroaryl (e.g., thienyl).

In some embodiments of Formula (1), X₁ and X₂ are hydrogen, R₁ is alkyl (e.g., methyl), and R₂ is hydrogen, A is —CH(R₁₀)—, and R₁₀ is heteroaryl (e.g., thienyl).

Isoquinoline compounds that may be used in compositions and methods described herein include compounds of Formula (1a) which has a tautomeric form, also shown here for clarification purposes:

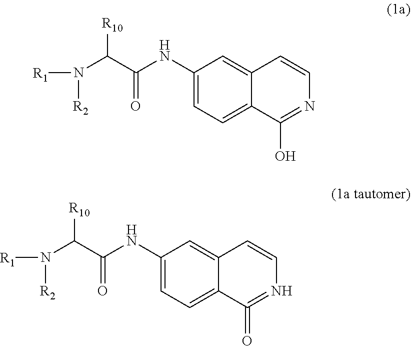

or a pharmaceutically acceptable salt thereof, wherein:

R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms; and

R₁₀ is selected from the group consisting of alkyl, alkenyl, alkynyl, amino, aryl, heteroaryl, cycloalkyl and heterocyclyl, any of which may be optionally substituted.

In some embodiments, R₁₀ is aryl (e.g., phenyl). In some embodiments, R₁₀ is heteroaryl (e.g., thienyl). In some embodiments, R₁ and R₂ are independently selected from the group consisting of hydrogen and methyl, or R₁ and R₂ are taken together with the nitrogen to which they are attached to form a heterocyclyl ring (e.g., pyrrolidone or piperidine).

Isoquinoline compounds that may be used in compositions and methods described herein include compounds of Formula (1b):

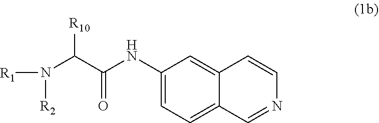

or a pharmaceutically acceptable salt thereof, wherein:

R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms; and

R₁₀ is selected from the group consisting of alkyl, alkenyl, alkynyl, amino, aryl, heteroaryl, cycloalkyl and heterocyclyl, any of which may be optionally substituted.

In some embodiments, R₁₀ is aryl (e.g., phenyl). In some embodiments, R₁₀ is aryl (e.g., phenyl) substituted with —CH₂—OC(O)—R^{a}, wherein R^{a} is optionally substituted aryl (e.g., phenyl, e.g., 2,4-dimethylphenyl). In some embodiments, R₁₀ is optionally substituted phenyl (e.g., 4-chlorophenyl). In some embodiments, R₁₀ is aryl (e.g., phenyl) substituted with —CH₂—OC(O)—NH—R^{b}, wherein R^{b} is optionally substituted aryl (e.g., phenyl, e.g., 2-chlorophenyl or 4-chlorophenyl or 4-methoxyphenyl) or wherein R^{b} is optionally substituted alkyl (e.g. C₁₋₄ alkyl, such as butyl). In some embodiments, R₁ and R₂ are independently selected from the group consisting of hydrogen and methyl, or R₁ and R₂ are taken together with the nitrogen to which they are attached to form a heterocyclyl ring (e.g., pyrrolidone or piperidine).

In embodiments, the compound of Formula (1) may be selected from the group consisting of:
(rac)-2-(dimethylamino)-N-(1-hydroxyisoquinolin-6-yl)-2-(thiophen-3-yl)acetamide;
(R)-2-(dimethylamino)-N-(1-hydroxyisoquinolin-6-yl)-2-(thiophen-3-yl)acetamide;
(S)-2-(dimethylamino)-N-(1-hydroxyisoquinolin-6-yl)-2-(thiophen-3-yl)acetamide;
(rac)-4-(3-amino-1-(isoquinolin-6-ylamino)-1-oxopropan-2-yl)benzyl 2,4-dimethylbenzoate;
(R)-4-(3-amino-1-(isoquinolin-6-ylamino)-1-oxopropan-2-yl)benzyl 2,4-dimethylbenzoate;
(S)-4-(3-amino-1-(isoquinolin-6-ylamino)-1-oxopropan-2-yl)benzyl 2,4-dimethylbenzoate;
(rac)-3-amino-2-(4-chlorophenyl)-N-(isoquinolin-6-yl)propanamide;
(R)-3-amino-2-(4-chlorophenyl)-N-(isoquinolin-6-yl)propanamide;
(S)-3-amino-2-(4-chlorophenyl)-N-(isoquinolin-6-yl)propanamide;

or a pharmaceutically acceptable salt thereof.

Compounds of Formula (1) may be synthesized by methods known in the art. For example, compounds may be synthesized using methods described in U.S. Patent Publication No. 2009/0186917, which is hereby incorporated by reference in its entirety.

In other embodiments, a compound according to Formula (5) is provided:

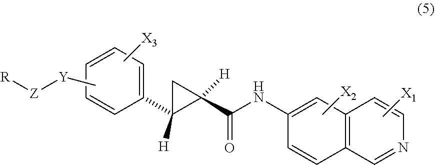

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;

Z is a direct bond or C₁₋₆ alkylene;

R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, the present disclosure provides a compound according to Formula (5a):

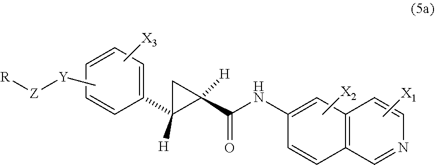

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —NR^{N1}(CR¹₂)ₘ—, —S(O)₂—, —C(O)—, —NR^{N1}C(O)O(CR¹₂)ₙ—, or C₁₋₆ alkylene;

Z is a direct bond or C₁₋₆ alkylene;

R is H, halogen, cyano, OR³, C₁₋₆ alkyl, amino, cycloalkyl, heterocyclyl, aryl, or heteroaryl, carbonyl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl, NH₂, SO₂-aryl, SO₂-heteroaryl, morpholine or piperidine, or forms a ring with R;

R¹ is H, F, or Me or forms a ring with R;

R³ is H or C₁₋₆ alkyl;

n is an integer from 0 to 6; and

m is an integer from 1 to 6.

In another embodiment, the present disclosure provides a compound according to Formula (5b):

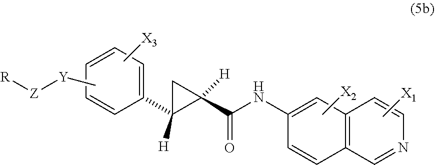

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O(CR¹₂)ₙ—, —(R)O— C₁₋₆ alkylene, aryl, or heteroaryl

Z is a direct bond or C₁₋₆ alkylene;

R is halogen, cyano, OR³, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl, NH₂, SO₂-aryl, SO₂-heteroaryl, morpholine or piperidine, or forms a ring with R;

R¹ is H, F, or Me or forms a ring with R;

R³ is H or C₁₋₆ alkyl;

n is an integer from 0 to 6; and

m is an integer from 1 to 6.

In another embodiment, the present disclosure provides a compound according to Formula (5c):

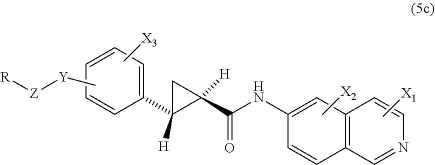

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O(CR¹₂)ₙ—, —(R)O— C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, carbonyl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl, NH₂, SO₂-aryl, SO₂-heteroaryl, morpholine or piperedine, or forms a ring with R;

R¹ is F, or Me or forms a ring with R;

R³ is H or C₁₋₆ alkyl;

n is an integer from 0 to 6; and

m is an integer from 1 to 6.

In another embodiment, the present disclosure provides a compound according to Formula (5d):

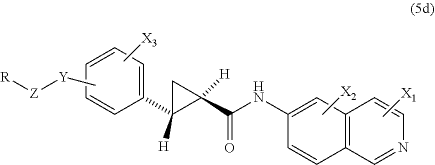

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O(CR¹₂)ₙ—, —(R)O— C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, carbonyl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl, NH₂, SO₂-aryl, SO₂-heteroaryl, morpholine or piperidine, or forms a ring with R;

R¹ is H, F, or Me or forms a ring with R;

R³ is H or C₁₋₆ alkyl;

n is 2, 3, 4, 5, or 6; and

m is an integer from 1 to 6.

In another embodiment, a compound according to Formula (6) is provided:

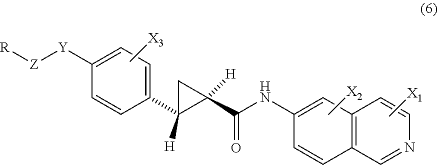

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, the compound according to Formula (7) is provided:

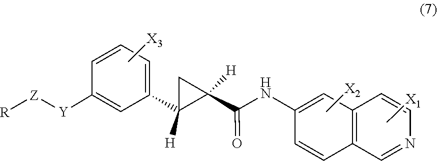

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl,

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, the compound according to Formula (8) is provided:

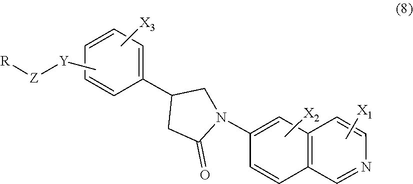

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R; R³ is H or C₁₋₆ alkyl;

n is an integer from 0 to 6; and

m is an integer from 1 to 6.

In another embodiment, a compound according to Formula (9) is provided:

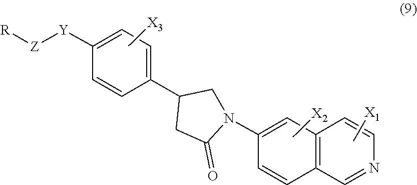

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, a compound according to Formula (10) is provided:

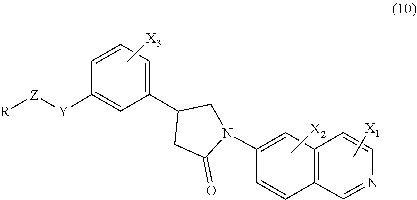

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, the compound according to Formula (11) is provided:

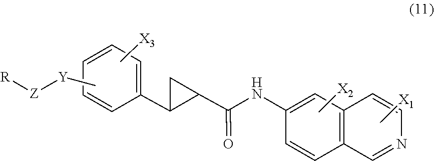

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, a compound according to Formula (12) is provided:

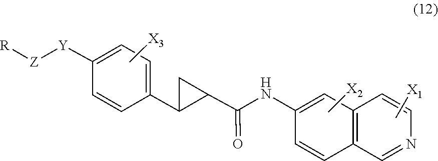

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In another embodiment, a compound according to Formula (13) is provided:

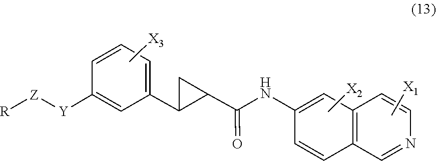

or tautomers, stereoisomers and pharmaceutically acceptable salts thereof,

wherein:

X₁, X₂ and X₃ are independently H, halogen, nitrile, hydroxyl, or C₁₋₆ alkyl;

Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;

Z is a direct bond or C₁₋₆ alkylene;

R is H, halogen, cyano, OR³, C₁₋₆ alkyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;

R^{N1} is H or C₁₋₆ alkyl or forms a ring with R;

R¹ is H, F, or Me or forms a ring with R;

R³ is H or C₁₋₆ alkyl;

n is an integer from 0 to 6; and

m is an integer from 1 to 6.

In embodiments for Formulas 5-13, one or more of X¹, X², and X³ are hydrogen. In embodiments for Formulas 5-13, X¹ is OH, CN, F, Br, Cl or CH₃. In embodiments for Formulas 5-13, X² is CN, F, Br, Cl or CH₃. In embodiments for Formulas 5-13, X³ is —CF₃, —OCH₃, CN, F, Br, Cl, OCF₃ or CH₃.

In embodiments for Formulas 5-13, R^{N1} is H. Alternatively, R^{N1} is C₁₋₆ alkyl, which may be substituted.

In embodiments for Formulas 5-13, R is amino. Alternatively, R is heteroaryl, heterocyclyl, or aryl, such as phenyl, pyridyl, piperidinyl, morpholino, thiophenyl, isoquinolinyl, quinolinyl or pyrrolidinyl. In embodiments for Formulas 5-13, R is H, C₁₋₆ alkyl, cycloalkyl, or heterocyclyl, or R may form a ring of 5 to 7 member atoms with R^{N1}, wherein the ring may contain up to 2 heteroatoms selected from N, O, and S, which may be saturated or unsaturated.

In embodiment for Formulas 5-13, Z is a direct bond. Alternatively, Z is C₁₋₆ alkylene, such as —CH₂—.

In embodiments for Formulas 5-13, Y is —NR^{N1}S(O)₂—. In embodiments for Formulas 5-13, Y is —NR^{N1}S(O)₂— and R^{N1} is H. In embodiments, Y is —NR^{N1}S(O)₂— and R is alkyl, aryl, heteroaryl, carbocyclyl, or heterocyclyl.

In embodiments for Formulas 5-13, Y is —NR^{N1}C(O)—. In embodiments for Formulas 5-13, Y is —NR^{N1}C(O)— and R is piperidinyl, morpholino, thiophenyl, isoquinolinyl, quinolinyl, thiofuranyl, benzothiophenyl, or pyrrolidinyl. In embodiments for Formulas 5-13, Y is —NR^{N1}C(O)— and R is H, C₁₋₆ alkyl, cycloalkyl, or heterocyclyl, or R may form a ring of 5 to 7 member atoms with R^{N1}, wherein the ring may contain up to 2 heteroatoms selected from N, O, and S, which may be saturated or unsaturated. In embodiments, Y is —NR^{N1}C(O)—, R is piperidinyl, R^{N1} is H, Z is —CH₂— and X¹, X², and X³ are H. In embodiments, Y is —NR^{N1}C(O)—R is isoquinolinyl, R^{N1} is H, Z is a direct bond, and X¹, X², and X³ are H.

In embodiments for Formulas 5-13, Y is —O(CR¹₂)ₙ—. In embodiments, Y is —O(CR¹₂)ₙ- and R is cycloalkyl, heterocyclyl, or heteroaryl, or R may form a ring of 5 to 7 member atoms with R¹, wherein the ring may contain up to 2 heteroatoms selected from N, O, and S, which may be saturated or unsaturated.

In embodiments for Formulas 5-13, Y is —NR^{N1}(CR¹₂)ₘ—. In embodiments for Formulas 5-13, Y is —NR^{N1}(CR¹₂)ₘ— and R is H, C₁₋₆ alkyl, cycloalkyl, heterocyclyl, aryl, or heteroaryl, or R may form a ring of 5 to 7 member atoms with R^{N1}, wherein the ring may contain up to 2 heteroatoms selected from N, O, and S, which may be saturated or unsaturated.

In embodiments for Formulas 5-13, Y is —C(O)O— or —OC(O)—. In embodiments for Formulas (V)-(XIII), Y is —C(O)O— or —OC(O)— and R is H, cycloalkyl, heterocyclyl, aryl, or heteroaryl and wherein Z is a direct bond.

In some embodiments of these aspects, Y is —NR^{N1}S(O)₂—, —NR^{N1}C(O)—, —NR^{N1}(CR¹₂)ₘ—, —S(O)₂—, —C(O)—, —NR^{N1}C(O)O(CR¹₂)ₙ—, or C₁₋₆ alkylene.

In some embodiments of these aspects, R is halogen, cyano, OR³, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated.

In some embodiments of these aspects, R¹ is F, or Me or forms a ring with R.

In some embodiments of these aspects, n is 2, 3, 4, 5, or 6.

Compounds according to the present disclosure include those shown in Table 1.

TABLE 1

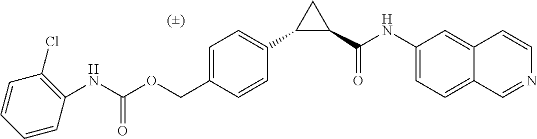 [SMILES: O=C(Nc1ccccc1Cl)OCc1ccc([C@@H]2C[C@H]2C(=O)Nc2ccc3cnccc3c2)cc1]

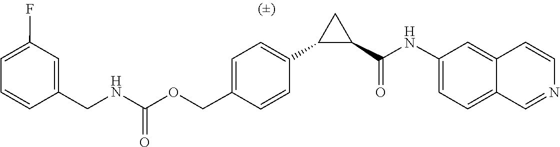 [SMILES: O=C(NCc1cccc(F)c1)OCc1ccc([C@@H]2C[C@H]2C(=O)Nc2ccc3cnccc3c2)cc1]

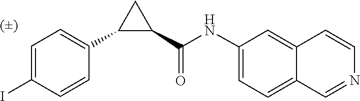 [SMILES: O=C(Nc1ccc2cnccc2c1)[C@@H]1C[C@H]1c1ccc(I)cc1]

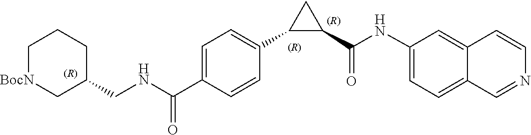

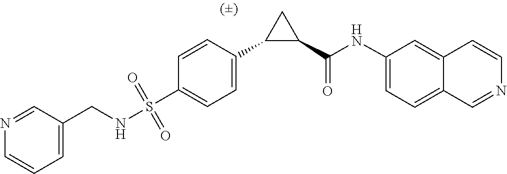 [SMILES: O=C(Nc1ccc2cnccc2c1)[C@@H]1C[C@H]1c1ccc(S(=O)(=O)NCc2cccnc2)cc1]

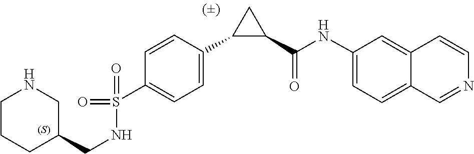

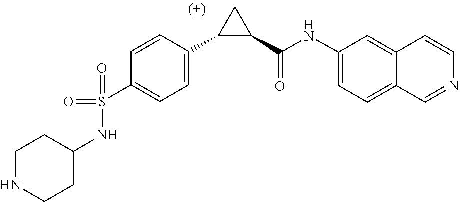 [SMILES: O=C(Nc1ccc2cnccc2c1)[C@@H]1C[C@H]1c1ccc(S(=O)(=O)NC2CCNCC2)cc1]

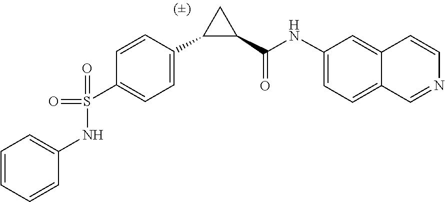 [SMILES: O=C(Nc1ccc2cnccc2c1)[C@@H]1C[C@H]1c1ccc(S(=O)(=O)Nc2ccccc2)cc1]

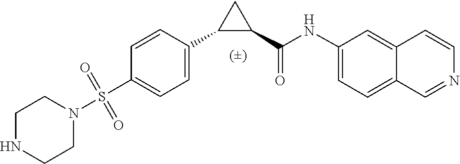 [SMILES: O=C(Nc1ccc2cnccc2c1)[C@@H]1C[C@H]1c1ccc(S(=O)(=O)N2CCNCC2)cc1]

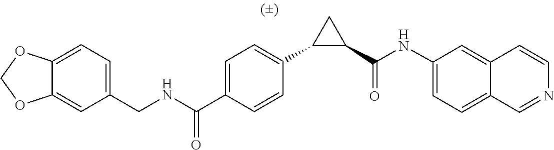 [SMILES: O=C(NCc1ccc2c(c1)OCO2)c1ccc([C@@H]2C[C@H]2C(=O)Nc2ccc3cnccc3c2)cc1]

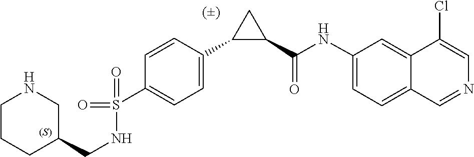

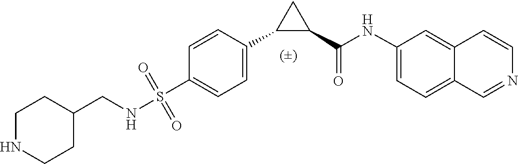 [SMILES: O=C(Nc1ccc2cnccc2c1)[C@@H]1C[C@H]1c1ccc(S(=O)(=O)NCC2CCNCC2)cc1]

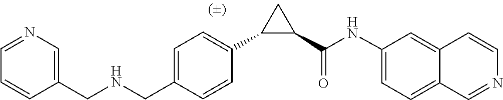 [SMILES: O=C(Nc1ccc2cnccc2c1)[C@@H]1C[C@H]1c1ccc(CNCc2cccnc2)cc1]

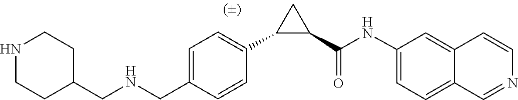 [SMILES: O=C(Nc1ccc2cnccc2c1)[C@@H]1C[C@H]1c1ccc(CNCC2CCNCC2)cc1]

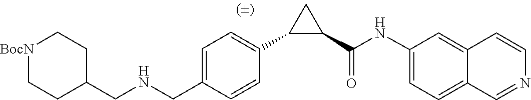 [SMILES: CC(C)(C)OC(=O)N1CCC(CNCc2ccc([C@@H]3C[C@H]3C(=O)Nc3ccc4cnccc4c3)cc2)CC1]

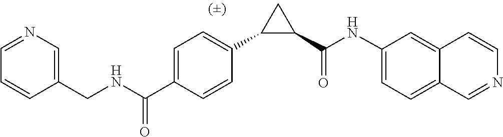 [SMILES: O=C(NCc1cccnc1)c1ccc([C@@H]2C[C@H]2C(=O)Nc2ccc3cnccc3c2)cc1]

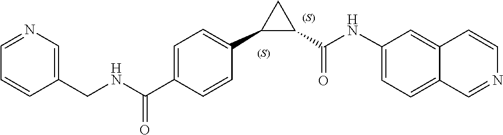

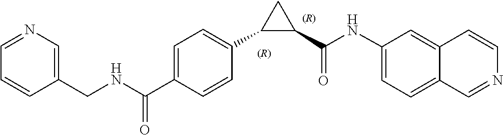

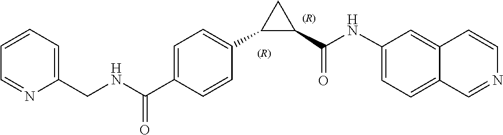

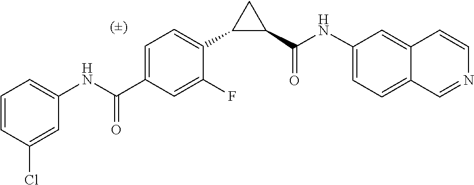 [SMILES: O=C(Nc1cccc(Cl)c1)c1ccc([C@@H]2C[C@H]2C(=O)Nc2ccc3cnccc3c2)c(F)c1]

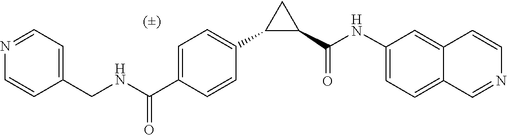 [SMILES: O=C(NCc1ccncc1)c1ccc([C@@H]2C[C@H]2C(=O)Nc2ccc3cnccc3c2)cc1]

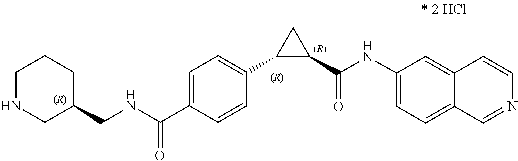

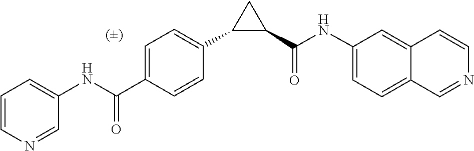 [SMILES: O=C(Nc1cccnc1)c1ccc([C@@H]2C[C@H]2C(=O)Nc2ccc3cnccc3c2)cc1]

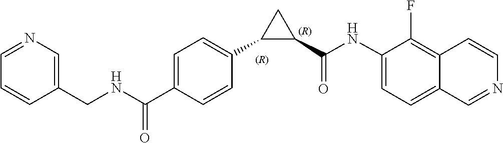

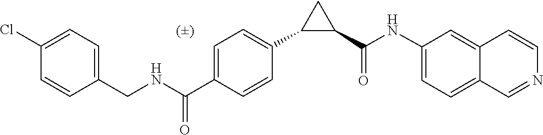 [SMILES: O=C(NCc1ccc(Cl)cc1)c1ccc([C@@H]2C[C@H]2C(=O)Nc2ccc3cnccc3c2)cc1]

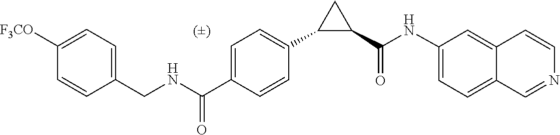 [SMILES: O=C(NCc1ccc(OC(F)(F)F)cc1)c1ccc([C@@H]2C[C@H]2C(=O)Nc2ccc3cnccc3c2)cc1]

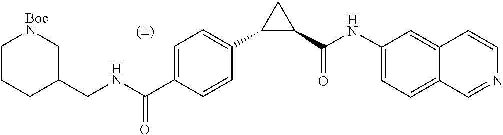 [SMILES: CC(C)(C)OC(=O)N1CCCC(CNC(=O)c2ccc([C@@H]3C[C@H]3C(=O)Nc3ccc4cnccc4c3)cc2)C1]

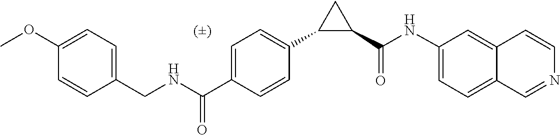 [SMILES: COc1ccc(CNC(=O)c2ccc([C@@H]3C[C@H]3C(=O)Nc3ccc4cnccc4c3)cc2)cc1]

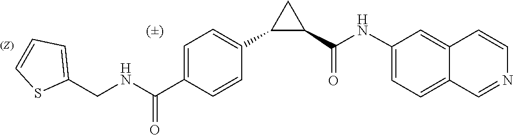 [SMILES: O=C(NCc1cccs1)c1ccc([C@@H]2C[C@H]2C(=O)Nc2ccc3cnccc3c2)cc1]

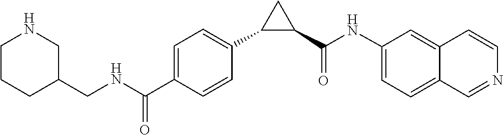 [SMILES: O=C(NCC1CCCNC1)c1ccc([C@@H]2C[C@H]2C(=O)Nc2ccc3cnccc3c2)cc1]

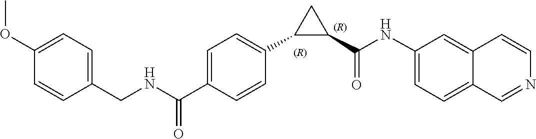

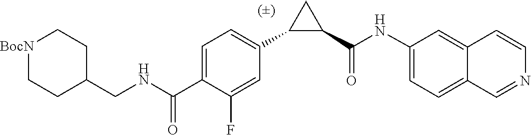 [SMILES: CC(C)(C)OC(=O)N1CCC(CNC(=O)c2ccc([C@@H]3C[C@H]3C(=O)Nc3ccc4cnccc4c3)cc2F)CC1]

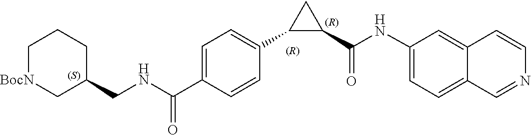

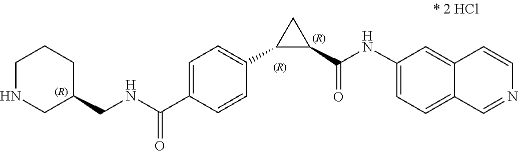

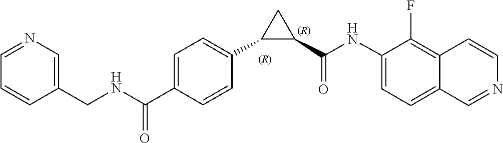

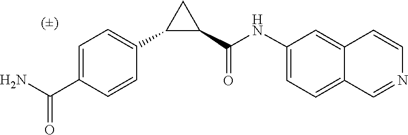 [SMILES: NC(=O)c1ccc([C@@H]2C[C@H]2C(=O)Nc2ccc3cnccc3c2)cc1]

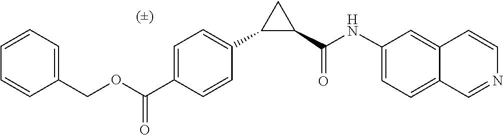 [SMILES: O=C(OCc1ccccc1)c1ccc([C@@H]2C[C@H]2C(=O)Nc2ccc3cnccc3c2)cc1]

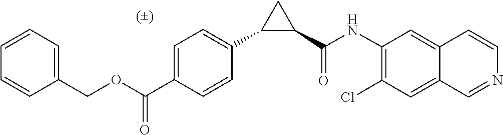 [SMILES: O=C(OCc1ccccc1)c1ccc([C@@H]2C[C@H]2C(=O)Nc2cc3ccncc3cc2Cl)cc1]

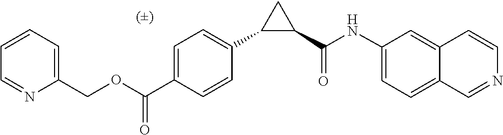 [SMILES: O=C(OCc1ccccn1)c1ccc([C@@H]2C[C@H]2C(=O)Nc2ccc3cnccc3c2)cc1]

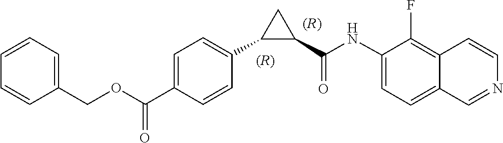

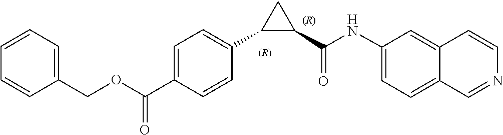

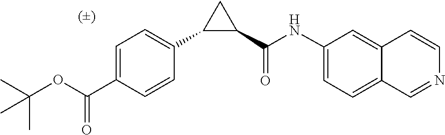 [SMILES: CC(C)(C)OC(=O)c1ccc([C@@H]2C[C@H]2C(=O)Nc2ccc3cnccc3c2)cc1]

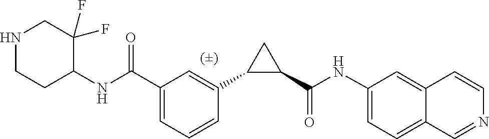 [SMILES: O=C(NC1CCNCC1(F)F)c1cccc([C@@H]2C[C@H]2C(=O)Nc2ccc3cnccc3c2)c1]

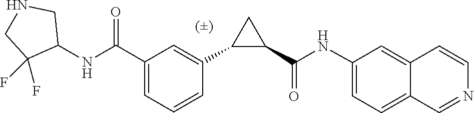 [SMILES: O=C(NC1CNCC1(F)F)c1cccc([C@@H]2C[C@H]2C(=O)Nc2ccc3cnccc3c2)c1]

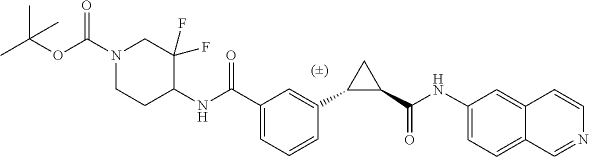 [SMILES: CC(C)(C)OC(=O)N1CCC(NC(=O)c2cccc([C@@H]3C[C@H]3C(=O)Nc3ccc4cnccc4c3)c2)C(F)(F)C1]

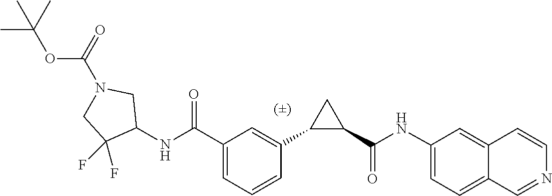 [SMILES: CC(C)(C)OC(=O)N1CC(NC(=O)c2cccc([C@@H]3C[C@H]3C(=O)Nc3ccc4cnccc4c3)c2)C(F)(F)C1]

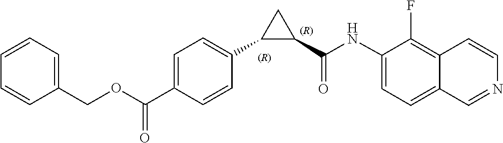

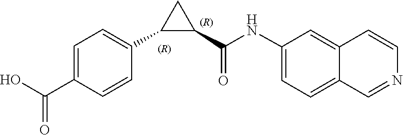

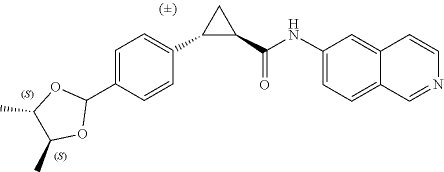

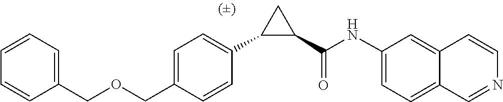 [SMILES: O=C(Nc1ccc2cnccc2c1)[C@@H]1C[C@H]1c1ccc(COCc2ccccc2)cc1]

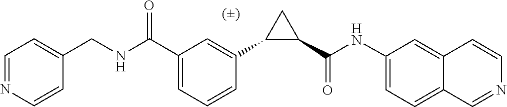 [SMILES: O=C(NCc1ccncc1)c1cccc([C@@H]2C[C@H]2C(=O)Nc2ccc3cnccc3c2)c1]

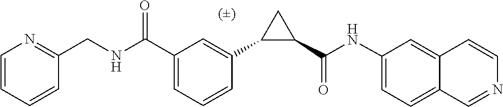 [SMILES: O=C(NCc1ccccn1)c1cccc([C@@H]2C[C@H]2C(=O)Nc2ccc3cnccc3c2)c1]

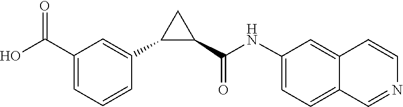 [SMILES: O=C(O)c1cccc([C@@H]2C[C@H]2C(=O)Nc2ccc3cnccc3c2)c1]

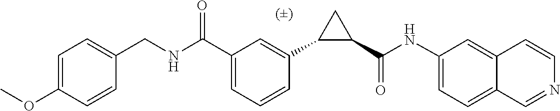 [SMILES: COc1ccc(CNC(=O)c2cccc([C@@H]3C[C@H]3C(=O)Nc3ccc4cnccc4c3)c2)cc1]

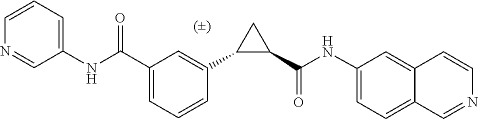 [SMILES: O=C(Nc1cccnc1)c1cccc([C@@H]2C[C@H]2C(=O)Nc2ccc3cnccc3c2)c1]

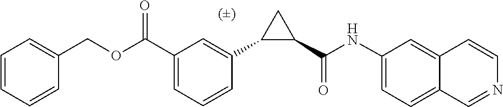 [SMILES: O=C(OCc1ccccc1)c1cccc([C@@H]2C[C@H]2C(=O)Nc2ccc3cnccc3c2)c1]

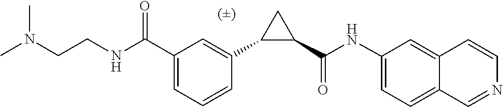 [SMILES: CN(C)CCNC(=O)c1cccc([C@@H]2C[C@H]2C(=O)Nc2ccc3cnccc3c2)c1]

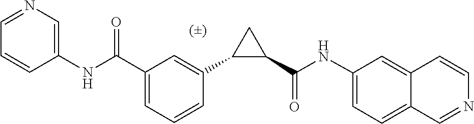 [SMILES: O=C(Nc1cccnc1)c1cccc([C@@H]2C[C@H]2C(=O)Nc2ccc3cnccc3c2)c1]

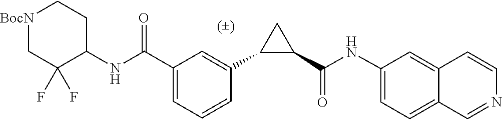 [SMILES: CC(C)(C)OC(=O)N1CCC(NC(=O)c2cccc([C@@H]3C[C@H]3C(=O)Nc3ccc4cnccc4c3)c2)C(F)(F)C1]

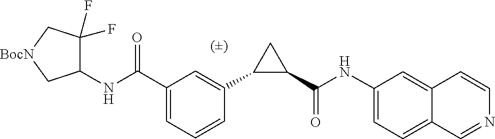 [SMILES: CC(C)(C)OC(=O)N1CC(NC(=O)c2cccc([C@@H]3C[C@H]3C(=O)Nc3ccc4cnccc4c3)c2)C(F)(F)C1]

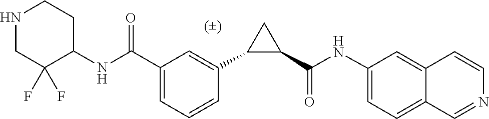 [SMILES: O=C(NC1CCNCC1(F)F)c1cccc([C@@H]2C[C@H]2C(=O)Nc2ccc3cnccc3c2)c1]

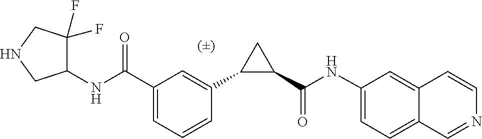 [SMILES: O=C(NC1CNCC1(F)F)c1cccc([C@@H]2C[C@H]2C(=O)Nc2ccc3cnccc3c2)c1]

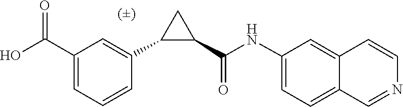 [SMILES: O=C(O)c1cccc([C@@H]2C[C@H]2C(=O)Nc2ccc3cnccc3c2)c1]

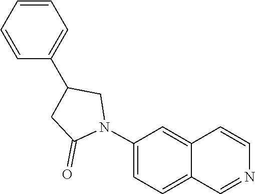 [SMILES: O=C1CC(c2ccccc2)CN1c1ccc2cnccc2c1]

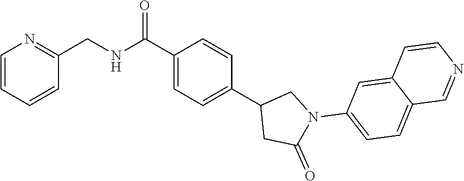 [SMILES: O=C(NCc1ccccn1)c1ccc(C2CC(=O)N(c3ccc4cnccc4c3)C2)cc1]

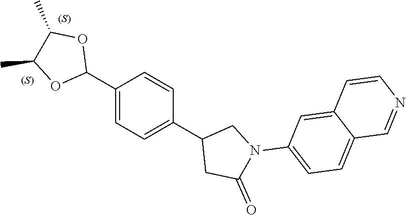

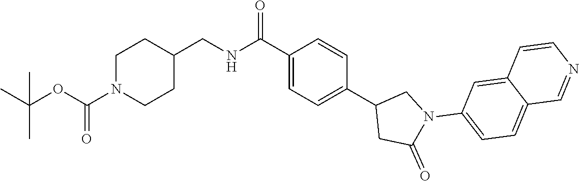 [SMILES: CC(C)(C)OC(=O)N1CCC(CNC(=O)c2ccc(C3CC(=O)N(c4ccc5cnccc5c4)C3)cc2)CC1]

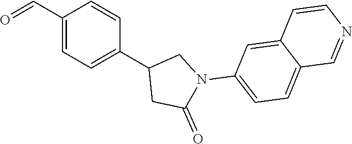 [SMILES: O=Cc1ccc(C2CC(=O)N(c3ccc4cnccc4c3)C2)cc1]

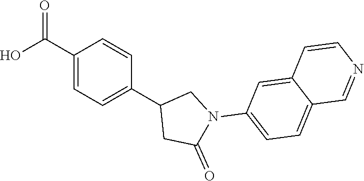 [SMILES: O=C(O)c1ccc(C2CC(=O)N(c3ccc4cnccc4c3)C2)cc1]

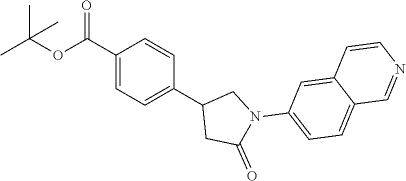 [SMILES: CC(C)(C)OC(=O)c1ccc(C2CC(=O)N(c3ccc4cnccc4c3)C2)cc1]

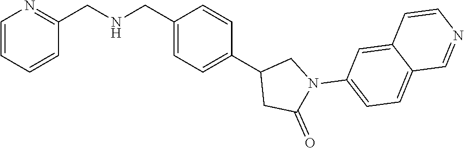 [SMILES: O=C1CC(c2ccc(CNCc3ccccn3)cc2)CN1c1ccc2cnccc2c1]

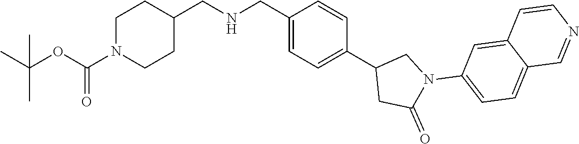 [SMILES: CC(C)(C)OC(=O)N1CCC(CNCc2ccc(C3CC(=O)N(c4ccc5cnccc5c4)C3)cc2)CC1]

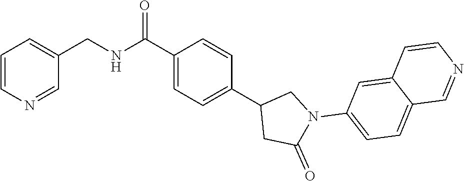 [SMILES: O=C(NCc1cccnc1)c1ccc(C2CC(=O)N(c3ccc4cnccc4c3)C2)cc1]

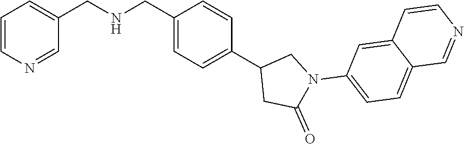 [SMILES: O=C1CC(c2ccc(CNCc3cccnc3)cc2)CN1c1ccc2cnccc2c1]

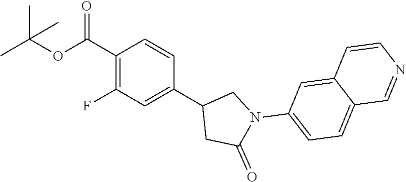 [SMILES: CC(C)(C)OC(=O)c1ccc(C2CC(=O)N(c3ccc4cnccc4c3)C2)cc1F]

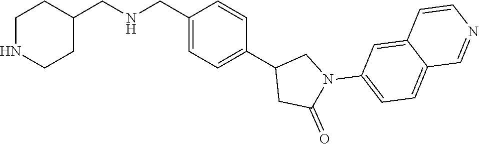 [SMILES: O=C1CC(c2ccc(CNCC3CCNCC3)cc2)CN1c1ccc2cnccc2c1]

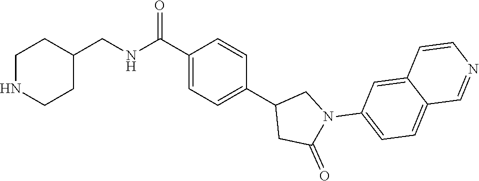 [SMILES: O=C(NCC1CCNCC1)c1ccc(C2CC(=O)N(c3ccc4cnccc4c3)C2)cc1]

Compounds according to the disclosure also include:

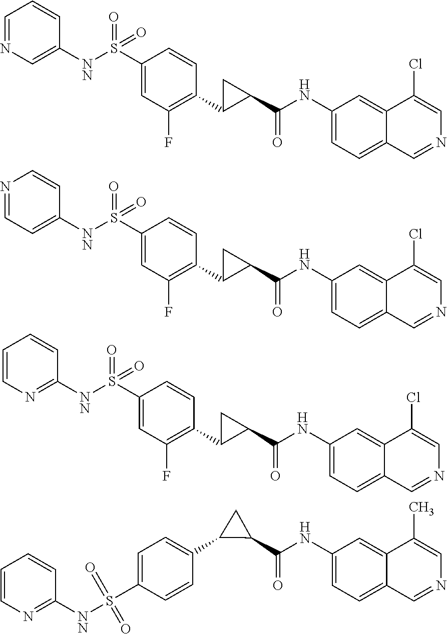

or a pharmaceutically acceptable salt thereof.

In another aspect, provided herein are compounds of Formula (14):

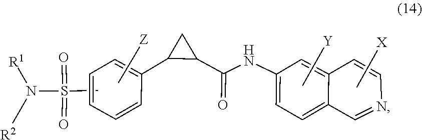

or a pharmaceutically acceptable salt thereof,
wherein,
R¹ is H, —C₁₋₆ alkyl, —C₁₋₆ haloalkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
R² is H, —C₁₋₆ alkyl, —C₁₋₆ haloalkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
or R¹ and R², together with the nitrogen to which they are attached, form a -heterocycle or a heterocycle substituted with —C₁₋₆ alkyl;
R³ is H, C₁₋₆ alkyl or —C₁₋₆ haloalkyl;
R⁴ is H, C₁₋₆ alkyl or —C₁₋₆ haloalkyl;
X is H, C₁₋₆ alkyl, —C₁₋₆ haloalkyl, halogen or hydroxyl;
Y is H, C₁₋₆ alkyl, —C₁₋₆ haloalkyl, halogen or hydroxyl; and
Z is H, C₁₋₆ alkyl, —C₁₋₆ haloalkyl, halogen or hydroxyl.

In an embodiment, the compound of Formula (14) is a compound of Formula (14a):

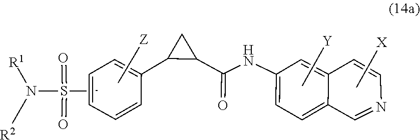

or a pharmaceutically acceptable salt thereof,
wherein,
R¹ is H, —C₁₋₆ alkyl, —C₁₋₆ haloalkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
R² is H, —C₁₋₆ alkyl, —C₁₋₆ haloalkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
or R¹ and R², together with the nitrogen to which they are attached, form a -heterocycle;
R³ is H, C₁₋₆ alkyl or —C₁₋₆ haloalkyl;
R⁴ is H, C₁₋₆ alkyl or —C₁₋₆ haloalkyl;
X is H, C₁₋₆ alkyl, —C₁₋₆ haloalkyl, halogen or hydroxyl;
Y is H, C₁₋₆ alkyl, —C₁₋₆ haloalkyl, halogen or hydroxyl; and
Z is H, C₁₋₆ alkyl, —C₁₋₆ haloalkyl, halogen or hydroxyl.

In an embodiment, the compound of Formula (14) is a compound of Formula (14b):

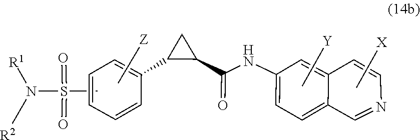

or a pharmaceutically acceptable salt thereof.

In an embodiment, the compound is of Formula (15):

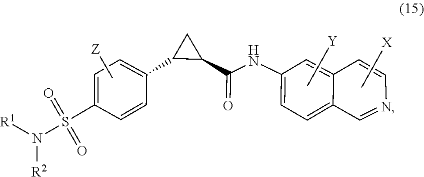

or a pharmaceutically acceptable salt thereof.

In an embodiment, the compound is of Formula (16):

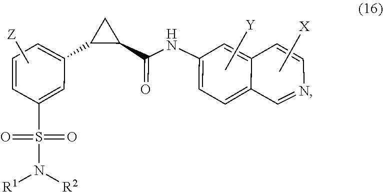

or a pharmaceutically acceptable salt thereof.

In an embodiment, the compound is of Formula (17):

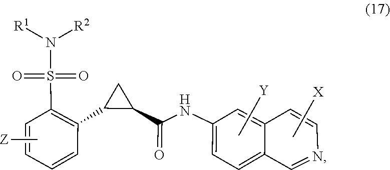

or a pharmaceutically acceptable salt thereof.
In an embodiment, the compound is of Formula (18):

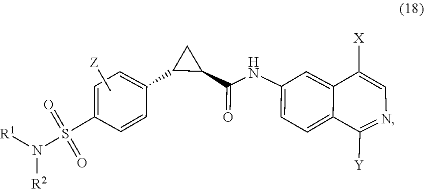

or a pharmaceutically acceptable salt thereof.

In an embodiment, the compound is of Formula (19):

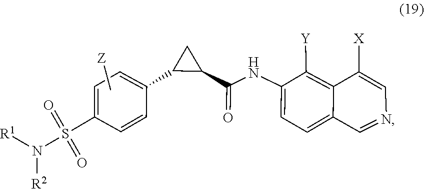

or a pharmaceutically acceptable salt thereof.

In an embodiment, the compound is of Formula (20):

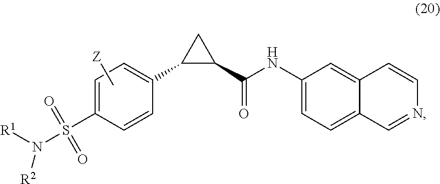

or a pharmaceutically acceptable salt thereof.

In an embodiment, the compound is of Formula (21):

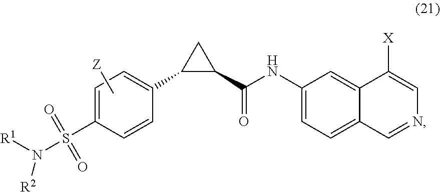

or a pharmaceutically acceptable salt thereof.

In an embodiment, the compound is of Formula (22)

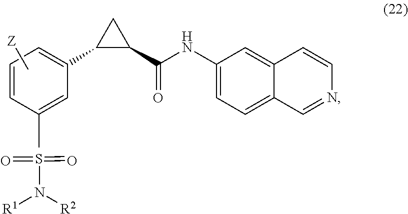

or a pharmaceutically acceptable salt thereof.

In an embodiment, the compound is of Formula (23):

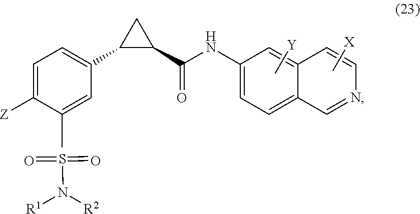

or a pharmaceutically acceptable salt thereof.

In an embodiment, the compound is of Formula (24)

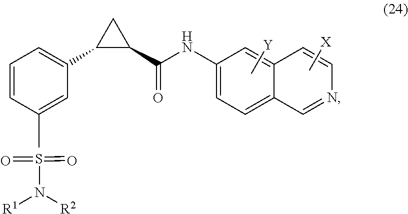

or a pharmaceutically acceptable salt thereof.

In an embodiment, the compound is of Formula (25):

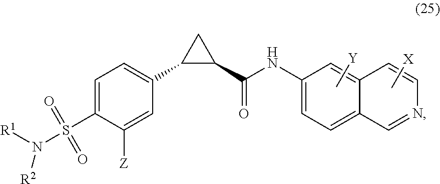

or a pharmaceutically acceptable salt thereof.

In an embodiment, the compound is of Formula (26):

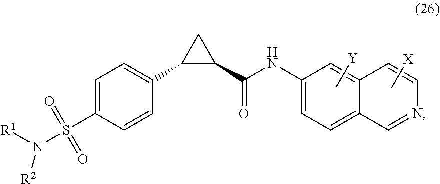

or a pharmaceutically acceptable salt thereof.

In some embodiments of the Formulae provided herein, the compound is trans (±) with respect to the stereocenters of the cyclopropyl ring of the compound. In some embodiments of the Formulae provided herein, the compound is (R,R) with respect to the stereocenters of the cyclopropyl ring of the compound. In some embodiments of the Formulae provided herein, the compound is (S,S) with respect to the stereocenters of the cyclopropyl ring of the compound.

In some embodiments of the Formulae provided herein,

R¹ is H, —C₁₋₆ alkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
R² is H, —C₁₋₆ alkyl, aryl, heteroaryl, —(C₁₋₆ alkyl)-pyridinyl, —(C₁₋₆ alkyl)-N(R³)R⁴, —(C₁₋₆ alkyl)-heterocyclyl or heterocycloalkyl;
or R¹ and R², together with the nitrogen to which they are attached, form a heterocycle;
R³ is H or C₁₋₆ alkyl;
R⁴ is H or C₁₋₆ alkyl;
X is H, C₁₋₆ alkyl, halogen or hydroxyl;
Y is H, C₁₋₆ alkyl, halogen or hydroxyl; and
Z is H, C₁₋₆ alkyl, halogen or hydroxyl.

In some embodiments of the Formulae provided herein,

R¹ is H, —C₁₋₄ alkyl, aryl, heteroaryl, —(C₁₋₄ alkyl)-pyridinyl, —(C₁₋₄ alkyl)-N(R³)R⁴, —(C₁₋₄ alkyl)-heterocyclyl or heterocycloalkyl;
R² is H, —C₁₋₄ alkyl, aryl, heteroaryl, —(C₁₋₄ alkyl)-pyridinyl, —(C₁₋₄ alkyl)-N(R³)R⁴, —(C₁₋₄ alkyl)-heterocyclyl or heterocycloalkyl;
or R¹ and R², together with the nitrogen to which they are attached, form a heterocycle;
R³ is H or C₁₋₄ alkyl;
R⁴ is H or C₁₋₄ alkyl;
X is H, C₁₋₄ alkyl, halogen or hydroxyl;
Y is H, C₁₋₄ alkyl, halogen or hydroxyl; and
Z is H, C₁₋₄ alkyl, halogen or hydroxyl.

In some embodiments of the Formulae provided herein,

R¹ is H, phenyl, pyridinyl, —(C₁₋₆ alkyl)-pyridinyl, —(C₁₋₆ alkyl)-N(R³)R⁴, —(C₁₋₆ alkyl)-heterocyclyl or heterocycloalkyl; and
R² is H, phenyl, pyridinyl, —(C₁₋₆ alkyl)-pyridinyl, —(C₁₋₆ alkyl)-N(R³)R⁴, —(C₁₋₆ alkyl)-heterocyclyl or heterocycloalkyl.

In some embodiments of the Formulae provided herein,

### R¹ is H; and

R² is H, phenyl, pyridinyl, —(C₁₋₆ alkyl)-pyridinyl, —(C₁₋₆ alkyl)-N(R³)R⁴, —(C₁₋₆ alkyl)-heterocyclyl or heterocycloalkyl.

In some embodiments of the Formulae provided herein, R¹ is H or —C₁₋₆ alkyl.

In some embodiments of the Formulae provided herein, R¹ is —C₁₋₆ alkyl.

In some embodiments of the Formulae provided herein, R¹ is H.

In some embodiments of the Formulae provided herein, R² is phenyl, pyridinyl, —(C₁₋₆ alkyl)-pyridinyl, —(C₁₋₆ alkyl)-N(R³)R⁴, —(C₁₋₆ alkyl)-heterocyclyl or heterocycloalkyl.

In some embodiments of the Formulae provided herein, pyridinyl is 2-pyridinyl.

In some embodiments of the Formulae provided herein, pyridinyl is 3-pyridinyl.

In some embodiments of the Formulae provided herein, pyridinyl is 4-pyridinyl.

In some embodiments of the Formulae provided herein, R² is phenyl, pyridinyl, —(C₁₋₆ alkyl)-pyridinyl, —(C₁₋₆ alkyl)-heterocyclyl or heterocycloalkyl.

In some embodiments of the Formulae provided herein, R² is —(C₁₋₆ alkyl)-N(R³)R⁴, —(C₁₋₆ alkyl)-heterocyclyl or heterocycloalkyl;

In some embodiments of the Formulae provided herein, R² is phenyl, pyridinyl, —(C₁₋₆ alkyl)-pyridinyl.

In some embodiments of the Formulae provided herein, R² is pyridinyl.

In some embodiments of the Formulae provided herein, R¹ and R², together with the nitrogen to which they are attached, form a heterocyclyl containing six ring atoms.

In some embodiments of the Formulae provided herein, R¹ and R², together with the nitrogen to which they are attached, form a heterocyclyl containing six ring atoms, wherein one or two of the ring atoms are, independently, O, S or N.

In some embodiments of the Formulae provided herein, R¹ and R², together with the nitrogen to which they are attached, form a heterocyclyl containing six ring atoms, wherein one or two of the ring atoms are N.

In some embodiments of the Formulae provided herein, R³ and R⁴ are H.

In some embodiments of the Formulae provided herein, R³ and R⁴ are, independently, C₁₋₆ alkyl.

In some embodiments of the Formulae provided herein, R³ is H, and R⁴ is C₁₋₆ alkyl.

In some embodiments of the Formulae provided herein, X, Y and Z are H.

In some embodiments of the Formulae provided herein,

X is C₁₋₆ alkyl, halogen or hydroxyl; and

### Y and Z are H.

In some embodiments of the Formulae provided herein,

X is halogen; and

### Y and Z are H.

In some embodiments of the Formulae provided herein, X is C₁₋₆ alkyl, halogen or hydroxyl.

In some embodiments of the Formulae provided herein, X is methyl, ethyl, CF₃, CHF₂ or CH₂F.

In some embodiments of the Formulae provided herein, Y is methyl, ethyl, CF₃, CHF₂ or CH₂F.

In some embodiments of the Formulae provided herein, Z is methyl, ethyl, CF₃, CHF₂ or CH₂F.

In some embodiments of the Formulae provided herein, X is halogen.

In some embodiments of the Formulae provided herein, X is F or Cl.

In some embodiments of the Formulae provided herein, X is Cl.

In some embodiments of the Formulae provided herein,

X is methyl or halogen;
Y is methyl or halogen; and
Z is methyl or halogen.

In some embodiments of the Formulae provided herein,

X is methyl, F or Cl;
Y is methyl, F or Cl; and
Z is methyl, F or Cl.

In some embodiments of the Formulae provided herein,

X is halogen; and
Y is hydroxyl.

In some embodiments of the Formulae provided herein, Y is hydroxyl.

In some embodiments of the Formulae provided herein, Z is H or F.

In some embodiments, provided herein is a compound of Formula (27):

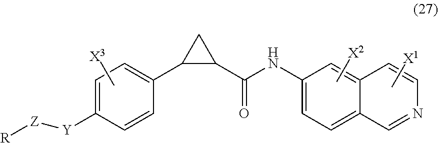

or a pharmaceutically acceptable salt thereof;
wherein
Y is —C₁₋₆-alkyl, —O—, —(CH₂)₁₋₂OC(O)N(H)—, —(CH₂)₁₋₂N(H)—, —C(O)N(H)— (i.e. an amide) or —C(O)O— (i.e. an ester);
X¹ is H, —OH, —C₁₋₃-alkyl (e.g., methyl), or halogen (e.g., F, Br or Cl);
X² is H or halogen (e.g., F, Cl or Br);
X³ is H or halogen (e.g., F, Cl or Br);
Z is a bond, —S(O)₂—, ethenyl, ethynyl, methylene, ethylene, or propylene, or Z, together with the nitrogen to which Z is attached forms a —C₂₋₆-heterocyloalkyl; and
R is —OH, —NH₂, —NH(C₁₋₃-alkyl), —N(C₁₋₃-alkyl)(C₁₋₃-alkyl), —C(O)O—(C₁₋₆-alkyl), —N(H)C(O)—(C₁₋₆-alkyl), —C₁₋₃-alkyl, pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂₋₆-heterocyloalkyl, oxydiaryl (e.g., oxydiphenyl, oxydinaphthalenyl, or phenoxynaphthalenyl), or R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl);
or Y and Z together are a bond, and R is —OH, —NH₂, —NH(C₁₋₃-alkyl), —N(C₁₋₃-alkyl)(C₁₋₃-alkyl), —N(H)C(O)—(C₁₋₆-alkyl), —C(O)NH₂, —C₁₋₃-alkyl, pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂₋₆-heterocyloalkyl, oxydiaryl (e.g., oxydiphenyl, oxydinaphthalenyl, or phenoxynaphthalenyl), or R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl).

In some embodiments, Y is —C(O)N(H)— (i.e. an amide). In some embodiments, Y is —C(O)O— (i.e. an ester). In some embodiments, Y is —C₁₋₆-alkyl. In some embodiments, Y is —(CH₂)₁₋₂OC(O)N(H)— or —(CH₂)₁₋₂N(H)—. In some embodiments, Y is —O—.

In some embodiments, Y and Z together are a bond.

In some embodiment, X¹ is —OH, methyl, F, Br or Cl.

In some embodiments, X² is F, Cl or Br.

In some embodiments, X³ is F, Cl or Br.

In some embodiments, Z is —S(O)₂—. In some embodiments, Z is a bond. In some embodiments, Z is ethenyl, ethynyl, methylene, ethylene, or propylene. In some embodiments, Z, together with the nitrogen to which Z is attached forms a —C₂₋₆-heterocyloalkyl.

In some embodiments, R is —OH, —NH₂, —NH(C₁₋₃-alkyl), —N(C₁₋₃-alkyl)(C₁₋₃-alkyl), —C(O)O—(C₁₋₆-alkyl), —N(H)C(O)—(C₁₋₆-alkyl), or —C₁₋₃-alkyl. In some embodiments, R is —OH, —NH₂, —NH(C₁₋₃-alkyl), —N(C₁₋₃-alkyl)(C₁₋₃-alkyl), —N(H)C(O)—(C₁₋₆-alkyl), or —C₁₋₃-alkyl. In some embodiments, R is pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂₋₆-heterocyloalkyl, oxydiaryl (e.g., oxydiphenyl, oxydinaphthalenyl, or phenoxynaphthalenyl). In some embodiments, R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl).

In some embodiments, provided is a compound of Formula (28):

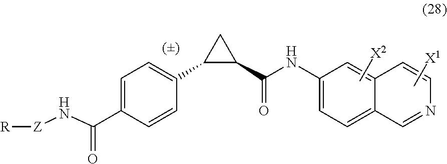

or a pharmaceutically acceptable salt thereof.

In some embodiments, provided is a compound of Formula (29):

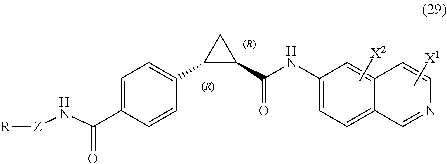

or a pharmaceutically acceptable salt thereof.

In some embodiments, provided is a compound of Formula (30):

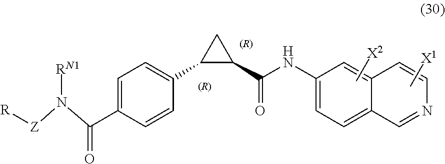

or a pharmaceutically acceptable salt thereof;
wherein
R—Z—N(R^{N1}) is —C₂₋₆-heterocyloalkyl unsubstituted or substituted with one or more of —C₁₋₆-alkyl, halo, phenyl, halophenyl, benzyl, halobenzyl, pyridinyl, carbonyl, monohalomethyl, dihalomethyl, trihalomethyl, or —C(O)O—(C₁₋₆-alkyl).

In some embodiments, provided is a compound of Formula (31):

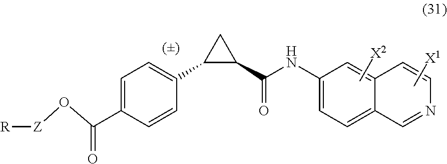

or a pharmaceutically acceptable salt thereof.

In some embodiments, provided is a compound of Formula (32):

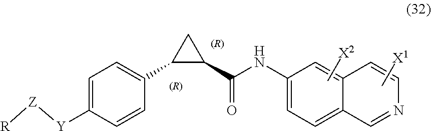

or a pharmaceutically acceptable salt thereof.

In some embodiments, provided is a compound of Formula (33):

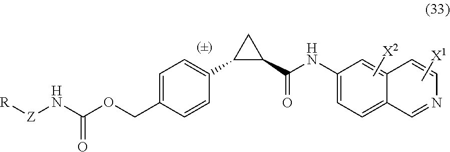

or a pharmaceutically acceptable salt thereof.

In some embodiments, provided is a compound of Formula (34):

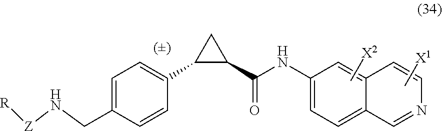

or a pharmaceutically acceptable salt thereof.

In some embodiments, provided is a compound of Formula (35):

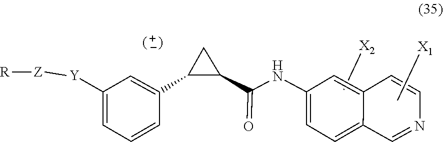

or a pharmaceutically acceptable salt thereof.

In some embodiments, provided is a compound of Formula (36):

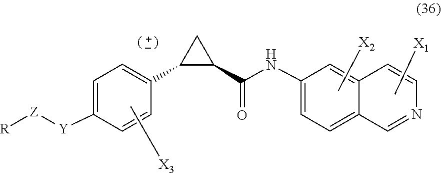

or a pharmaceutically acceptable salt thereof.

In some embodiments, provided is a compound of Formula (37):

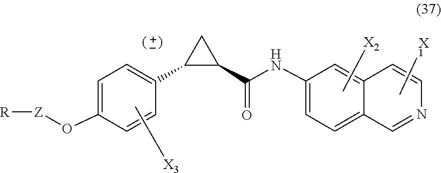

or a pharmaceutically acceptable salt thereof.

In another aspect, provided herein are compounds of Formula (38):

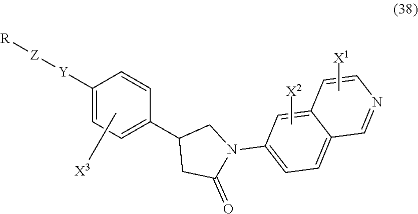

or a pharmaceutically acceptable salt thereof;
wherein
Y is —(CH₂)₁₋₂N(H)— or —C(O)N(H)— (i.e. an amide);
X¹ is H, —OH, —C₁₋₃-alkyl (e.g., methyl), or halogen (e.g., F, Br or Cl);
X² is H or halogen (e.g., F, Cl or Br);
X³ is H, —C₁₋₃-alkyl (e.g., methyl), or halogen (e.g., F, Cl or Br);
Z is a bond, ethenyl, ethynyl, methylene, ethylene, or propylene, or Z, together with the nitrogen to which Z is attached forms a —C₂₋₆-heterocyloalkyl; and
R is —OH, —NH₂, —NH(C₁₋₃-alkyl), —N(C₁₋₃-alkyl)(C₁₋₃-alkyl), —C(O)O—(C₁₋₆-alkyl), —N(H)C(O)—(C₁₋₆-alkyl), —C₁₋₃-alkyl, pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂-g-heterocyloalkyl, oxydiaryl (e.g., oxydiphenyl, oxydinaphthalenyl, or phenoxynaphthalenyl), or R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl); and
or Y and Z together are a bond, and R is —OH, —NH₂, —NH(C₁₋₃-alkyl), —N(C₁₋₃-alkyl)(C₁₋₃-alkyl), —N(H)C(O)—(C₁₋₆-alkyl), —C(O)NH₂, —C₁₋₃-alkyl, pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂₋₆-heterocyloalkyl, oxydiaryl (e.g., oxydiphenyl, oxydinaphthalenyl, or phenoxynaphthalenyl), or R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl).

In some embodiments, Y is —C(O)N(H)— (i.e. an amide). In some embodiments, Y is —C(O)O— (i.e. an ester). In some embodiments, Y is —C₁₋₆-alkyl. In some embodiments, Y is —(CH₂)₁₋₂OC(O)N(H)— or —(CH₂)₁₋₂N(H)—. In some embodiments, Y is —O—.

In some embodiments, Y and Z together are a bond.

In some embodiment, X¹ is —OH, methyl, F, Br or Cl.

In some embodiments, X² is F, Cl or Br.

In some embodiments, X³ is F, Cl or Br.

In some embodiments, Z is —S(O)₂—. In some embodiments, Z is a bond. In some embodiments, Z is ethenyl, ethynyl, methylene, ethylene, or propylene. In some embodiments, Z, together with the nitrogen to which Z is attached forms a —C₂₋₆-heterocyloalkyl.

In some embodiments, R is —OH, —NH₂, —NH(C₁₋₃-alkyl), —N(C₁₋₃-alkyl)(C₁₋₃-alkyl), —C(O)O—(C₁₋₆-alkyl), —N(H)C(O)—(C₁₋₆-alkyl), or —C₁₋₃-alkyl. In some embodiments, R is —OH, —NH₂, —NH(C₁₋₃-alkyl), —N(C₁₋₃-alkyl)(C₁₋₃-alkyl), —N(H)C(O)—(C₁₋₆-alkyl), or —C₁₋₃-alkyl. In some embodiments, R is pyridinyl, phenyl, halophenyl, methoxyphenyl, monohalomethoxyphenyl, diihalomethoxyphenyl, trihalomethoxyphenyl, monohalomethyl, dihalomethyl, trihalomethyl, thienyl, halothienyl, thiazolyl, benzothiophenyl, isoquinolinyl, —C₂₋₆-heterocyloalkyl, oxydiaryl (e.g., oxydiphenyl, oxydinaphthalenyl, or phenoxynaphthalenyl). In some embodiments, R is —C₂₋₆-heterocyloalkyl unsubstituted or substituted with one or more of —C₁₋₆-alkyl, halo, benzyl, halobenzyl, pyridinyl, carbonyl, monohalomethyl, dihalomethyl, trihalomethyl, or —C(O)O—(C₁₋₆-alkyl).

In some embodiments, provided is a compound of Formula (39):

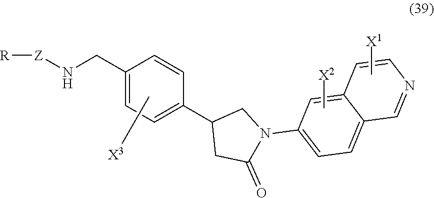

or a pharmaceutically acceptable salt thereof.

In some embodiments, provided is a compound of Formula (40):

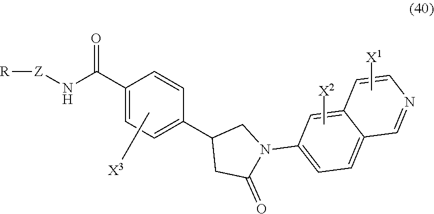

or a pharmaceutically acceptable salt thereof.

Compounds can include those presented in Table 1, Table 2, Table 3, Table 4, Table 5, Table 6, Table 7, Table 8, Table 9, Table 10, Table 11, Table 12, Table 13, Table 14, Table 15, Table 16, Table 17 or Table 18 of WO/2018183911 are also included and incorporated by reference. Further, pharmaceutically acceptable salts of the compounds in Table 1, Table 2, Table 3, Table 4, Table 5, Table 6, Table 7, Table 8, Table 9, Table 10, Table 11, Table 12, Table 13, Table 14, Table 15, Table 16, Table 17 or Table 18 of WO20/18183911 are also included and incorporated by reference.

In some embodiments, compounds in which an isoquinoline compound has ROCK inhibitory activity as well as JAK inhibitory activity has a formula:

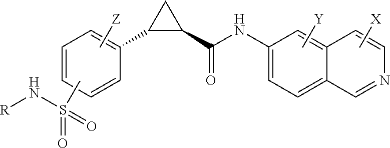

or a pharmaceutically acceptable salt thereof,

wherein:

ortho, meta or para substituted sulfonamides with a central phenyl ring
Y, X, Z are each independently H, halo (Br, Cl, F), lower alkyl (Me, Et, iPr, Pr, nBu, iBu, sBu, cyclopropyl), —CHF₂, —CF₃, —OCF₃, —CH₂CF₃, —CN, —CH₂CN, —NH₂, —OH, —CH₂OH, or —CH₂NH₂, and
R is hydrogen, alkyl, alkenyl, alkynyl, cycloalkyl, aryl, heteroaryl, heteroalkyl.

Compounds above may be synthesized according to methods in deLong, MA; Sturdivant, JM; Lichorowic, CL; Kornilov, A. Aryl Cyclopropyl Aminoisoquinolinyl Amide Compounds US Patent Application US2018-0327381 A1 published Nov. 15, 2018, which is incorporated into this application in its entirety.

Compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. The enantiomers also may be obtained from kinetic resolution of the racemate of corresponding esters using lipase enzymes.

Except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₃-alkyl or propyl includes n-propyl and iso-propyl; C₄-alkyl or butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hydroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

### Salts

A compound described herein can be in the form of a salt, e.g., a pharmaceutically acceptable salt. The term “pharmaceutically acceptable salt” includes salts of the active compounds that are prepared with relatively nontoxic acids or bases, depending on the particular substituents found on the compounds described herein. Neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure. Examples of pharmaceutically acceptable salts are discussed in Berge et al, 1977, “Pharmaceutically Acceptable Salts.” J. Pharm. Sci. Vol. 66, pp. 1-19.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations. Examples of suitable organic cations include, but are not limited to, ammonium ion (i.e., NH₄⁺) and substituted ammonium ions (e.g., NH₃R₁⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine.

If the compound is cationic, or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Chemically Protected Forms

It may be convenient or desirable to prepare, purify, and/or handle an active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999). Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

A hydroxy group may be protected as an ether (—OR) or an ester (—OC(O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(O)CH₃, —OAc).

An aldehyde or ketone group may be protected as an acetal (RCH(OR)₂) or ketal (R₂C(OR)₂), respectively, in which the carbonyl group (R₂C═O) is converted to a diether (R₂C(OR)₂), by reaction with, for example, a primary alcohol. The aldehyde or ketone group is readily regenerated by hydrolysis using a large excess of water in the presence of acid.

An amine group may be protected, for example, as an amide (—NRC(O)R) or a urethane (—NRC(O)OR), for example, as: a methyl amide (—NHC(O)CH₃); a benzyloxy amide (—NHC(O)OCH₂C₆H₅, —NH-Cbz); as a t-butoxy amide (—NHC(O)OC(CH₃)₃, —NH-Boc); a 2-biphenyl-2-propoxy amide (—NHCO(O)C(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH— Teoc), as a 2,2,2-trichloroethyloxy amide (—NH-Troc), as an allyloxy amide (—NH— Alloc), as a 2(-phenylsulphonyl)ethyloxy amide (—NH—Psec); or, in suitable cases (e.g., cyclic amines), as a nitroxide radical (>N-0«).

A carboxylic acid group may be protected as an ester, for example, as: an alkyl ester (e.g., a methyl ester; a t-butyl ester); a haloalkyl ester (e.g., a haloalkyl ester); a trialkylsilylalkyl ester; or an arylalkyl ester (e.g., a benzyl ester; a nitrobenzyl ester); or as an amide, for example, as a methyl amide.

A thiol group may be protected as a thioether (—SR), for example, as: a benzyl thioether; an acetamidomethyl ether (—S—CH₂NHC(O)CH₃)

### Prodrugs and Other Modifications

In addition to salt forms, also provided are compounds that are in a prodrug form. Prodrugs of the compounds described herein are those compounds that readily undergo chemical changes under physiological conditions to provide the compounds described herein. Prodrugs can be converted to the compounds of the present invention by chemical or biochemical methods in an ex vivo environment. For example, prodrugs can be slowly converted to the compounds of the present invention when placed in a transdermal patch reservoir with or without a suitable enzyme or chemical reagent.

A compound described herein can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivalates or fatty acid substituents, conversion to carbamates, hydroxylation of aromatic rings, and heteroatom substitution in aromatic rings.

Compositions of the present disclosure may comprise safe and effective amounts of the subject compounds. As used herein, “safe and effective amount” means an amount of a compound sufficient to significantly induce a positive modification in the condition to be treated, but low enough to avoid serious side effects (at a reasonable benefit/risk ratio), within the scope of sound medical judgment. A safe and effective amount of a compound will vary with the particular condition being treated, the age and physical condition of the patient being treated, the severity of the condition, the duration of treatment, the nature of concurrent therapy, the particular pharmaceutically-acceptable carrier utilized, and like factors within the knowledge and expertise of the attending physician.

In embodiments, a composition may include a compound of formula (I), (Ia), (Ib), (II) or (III) at an amount of about 0.001% to about 2.0% w/v, e.g., about 0.01% to about 1.0% w/v. In embodiments, a compound of formula (I), (Ia), (Ib), (II) or (III) may be included in a composition at an amount of less than about 0.0025%, less than about 0.010%, less than about 0.015%, less than about 0.025%, less than about 0.05%, less than about 0.080%, less than about 0.10%, less than about 0.20%, less than about 0.40%, less than about 0.60%, less than about 0.80%, less than about 0.10%, less than about 0.5%, less than about 0.7%, less than about 1.0%, less than about 1.2%, less than about 1.4%, less than about 1.5%, less than about 1.6%, less than about 1.8, less than about 2.0%, at least about 0.0025%, at least about 0.010%, at least about 0.015%, at least about 0.020%, at least about 0.05%, at least about 0.075%, at least about 0.10%, at least about 0.20%, at least about 0.40%, at least about 0.60%, at least about 0.80, at least about 1.0%, at least about 1.2%, at least about 1.4%, at least about 1.6%, at least about 1.8%, at least about 2.0, about 0.0025%, about 0.010%, about 0.015%, about 0.03%, about 0.05%, about 0.10%, about 0.20%, about 0.40%, about 0.60%, about 0.80%, about 1.0%, about 1.2%, about 1.4%, about 1.6%, about 1.8%, or about 2.0%.

### Combination Therapies

The presently described compounds can be combined with other compounds to enhance treatment. In some embodiments, presently described compounds can be combined with other antiviral compounds to enhance treatment.

Compositions including there herein described compounds can also include one or more JAK inhibitors selected from tofacitinib, decernotinib, INCB18424, baricitinib, CYT387, GLPG0634, AC-430, axitinib, ruxolitinib, fibotinib, peficitinib or a combination thereof.

The described compounds are intended to be useful in combination with one or more additional compounds useful for treatment as described herein. These additional compounds may comprise compounds known to treat, prevent, or reduce the symptoms or effects of a virus. Such compounds include but are not limited to polymerase inhibitors, interferons, viral entry inhibitors, viral maturation inhibitors, literature-described capsid assembly modulators, reverse transcriptase inhibitors, a TLR-agonists, and other agents with distinct or unknown mechanisms that affect the life cycle and/or affect the consequences of viral infection.

In non-limiting examples, the compounds of the invention may be used in combination with one or more drugs (or a salt thereof) selected from:

reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);
interferons, including but not limited to interferon alpha (IFN-α), interferon lambda (IFN-λ), and interferon gamma (IFN-γ);
viral entry inhibitors;
viral maturation inhibitors;
literature-described capsid assembly modulators, such as, but not limited to BAY 41-4109;
reverse transcriptase inhibitors;

### a TLR-agonists; and

agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In one embodiment, the additional therapeutic agent is an interferon. The term “interferon” or “IFN” refers to any member the family of highly homologous species-specific proteins that inhibit viral replication and cellular proliferation, and modulate immune response. Human interferons are grouped into three classes; Type I, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds described herein can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is a reverse transcriptase inhibitor, and is at least one of Zidovudine, Didanosine, Zalcitabine, 2′,3′-dideoxyadenosine, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, cidofovir, Efavirenz, Nevirapine, Delavirdine, and Etravirine.

In one embodiment, the additional therapeutic agent is a TLR modulator or a TLR agonist, such as a TLR-7 agonist or TLR-9 agonist. In a further embodiment of the combination therapy, the TLR agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}methyl)phenyl]acetate).

In any of the methods provided herein, the method may further comprise administering to the individual at least one coronavirus vaccine, an interferon, or any combination thereof.

In another aspect, provided herein is method of treatment in an individual in need thereof, comprising reducing the viral load by administering to the individual a therapeutically effective amount of a compound as described herein alone or in combination with a reverse transcriptase inhibitor; and further administering to the individual a therapeutically effective amount of a coronavirus vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, cidofovir, Efavirenz, Nevirapine, Delavirdine, or Etravirine.

### Additional Components

Compositions of the present disclosure may further include one or more pharmaceutically acceptable excipients. For example, compositions may include additional, pharmaceutically acceptable components such as buffers, tonicity agents, chelating agents, sugars or sugar alcohols, viscosity enhancers and surfactants.

A buffer may comprise, for example, phosphate buffer, borate buffer, citrate buffer, maleate buffer, tartrate buffer, acetate buffer, tris(hydroxymethyl)aminomethane (TRIS), an amino acid buffer (e.g., glycine), combination buffers such as borate/phosphate buffer, citrate/phosphate buffer, and the like. In embodiments, a composition may include an amount of a buffer that is effective to provide a suitable buffering capacity to a composition. Other components of the compositions, while having other functions, may also affect the buffer capacity. For example, ethylenediaminetetraacetic acid (EDTA), often used as a chelating agent, can have an effect on the buffer capacity of a solution.

Compositions may include one or more tonicity agents, such that the composition may be isotonic with body fluids. A tonicity agent can be non-ionic or ionic. Non-ionic tonicity agents include sugars, sugar alcohols and other polyols, diols such as glycerol, mannitol, erythritol, and sugars such as dextrose. Other non-ionic tonicity agents such as polyethylene glycols, propylene glycol, which also function as co-solvents, can also be used. A tonicity agent can also be an ionic agent such as, for example, sodium chloride, potassium chloride, a balanced salt solution, sodium phosphate, or sodium citrate. For example, a non-ionic tonicity agent may be included in a composition at an amount of about 0.10 to about 20%, about 1.0 to about 10%, or about 2.0 to about 6.0%. An ionic tonicity agent may be included in a composition at an amount of about 0.10% to about 2.5%, about 0.25% to about 2.0%, or about 0.50% to about 1.0% w/v.

Compositions may also include one or more chelating agents or sequestering agents. A wide range of organic acids, amines or compounds which include an acid group and an amine function are capable of acting as chelating agents. For example, nitrilotriacetic acid, diethylenetriaminepentacetic acid, hydroxyethylethylenediaminetriacetic acid, 1,2-diaminocyclohexane tetraacetic acid, hydroxyethylaminodiacetic acid, ethylenediaminetetraacetic acid and its salts, polyphosphates, citric acid and its salts, tartaric acid and its salts, and the like and mixtures thereof, are useful as chelating agents. Ethylenediaminetetraacetic acid (EDTA) and its alkali metal salts, are suitable chelating agents, such as the disodium salt of EDTA (also known as disodium edetate). In embodiments, a chelating agent may be included in a composition at an amount of about 0.001% to about 0.25% w/v, about 0.005% to about 0.15% w/v, or about 0.01% to about 0.1% w/v. In embodiments, a composition may include a chelating agent in an amount effective to enhance the effectiveness of an antimicrobial component and/or to complex with metal ions.

Compositions may further include one or more preservatives. Suitable preservatives include, but are not limited to, sodium bisulfite, sodium bisulfate, sodium thiosulfate, ascorbate, benzalkonium chloride, benzododecinium bromide, chlorobutanol, thimerosal, phenylmercuric acetate, phenylmercuric borate, phenylmercuric nitrate, parabens such as methylparaben, ethylparaben and propylparaben, polyvinyl alcohol, benzyl alcohol, phenylethanol, sodium benzoate, sorbic acid, polyquaternium-1, and the like and mixtures thereof. In embodiments, a composition may include a preservative in amounts of 0.001 to about 1% or about 0.005 to about 0.10% w/v. In embodiments, a composition may include a preservative in an amount that is effective to inhibit microbial growth or contamination of the composition.

Compositions may additionally include a surfactant. Surfactants include non-ionic, anionic, amphoteric and zwitterionic surfactants. Exemplary surfactants include but are not limited to sodium lauryl sulfate, polyethoxylated sorbitan fatty acid esters, polyoxyethylene alkyl ethers, polyoxyethylene stearates (e.g., polyoxyethylene(40) stearate such as MYRJ-52), poloxamers, polaxamines, sorbitan fatty acid esters, polyethylene glycols (e.g., PEG-400), polyethoxylated alcohols, polyethoxylated castor oils (e.g., PEG-40 hydrogenated castor oil, such as Cremophor® RH 40), docusate sodium, quaternary ammonium compounds, medium and long chain fatty acids, sugar esters of fatty acids and glycerides of fatty acids, lecithin, polysorbate 80, phospholipids and sodium lauryl sulfate. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. Surfactants may be included in compositions at amounts of about 0.01% to about 5%, or about 0.1% to about 2% w/v.

Compositions may also include a viscosity enhancer, which may increase the resident time of a composition on the ocular surface. Exemplary viscosity enhancers include but are not limited to water soluble natural gums, cellulose-derived polymers and the like. Suitable natural gums include guar gum, gum tragacanth and the like. Suitable cellulose-derived viscosity inducing components include cellulose-derived polymers, such as sodium carboxymethylcellulose, hydroxypropyl cellulose, hydroxypropylmethyl cellulose, carboxymethyl cellulose, methyl cellulose, ethyl cellulose, hydroxyethyl cellulose and the like. Viscosity enhancers may be included in compositions at amounts of about 0.01% to about 5%, or about 0.1% to about 3% w/v.

Compositions described herein may also include a solvent. Compositions are typically aqueous, but may also include optional co-solvents. Suitable co-solvents include but are not limited to alcohols such as ethanol and isopropanol, ethylene glycol monoethyl ether, diethylene glycol monobutyl ether, diethylene glycol monoethyl ether, dimethylsulfoxide, dimethyl formamide, castor oil and combinations thereof. In addition to a compound of formula (I) or (II) a prostaglandin, and other optional components, the balance of a composition may comprise solvent.

pH

The pH of compositions can affect both stability of the compound and its efficacy. For example, higher pH may result in decomposition of a compound of formula (I), while lower pH may be irritating to the eye. In embodiments, the pH may be about 4.0 to about 7.0, or about 5.0 to about 6.0. In embodiments, a composition may have a pH of at least about 5.0, at least about 5.5, at least about 6.0, at least about 6.5, at least about 7.0, less than about 5.0, less than about 5.5, less than about 6.0, less than about 6.5, less than about 7.0, about 5.0, about 5.1, about 5.2, about 5.3, about 5.4, about 5.5, about 5.6, about 5.7, about 5.8, about 5.9, or about 6.0.

Composition pH can be adjusted with acid or base, if necessary. Any acid or base compatible with the components of the composition can be used. Exemplary acids include hydrochloric acid, citric acid, gluconic acid, lactic acid, acetic acid, and glycolic acid. Exemplary bases include sodium hydroxide, potassium hydroxide, and triethanolamine.

### Methods of Making Compositions

Compositions may be prepared using standard methods. In embodiments, composition components may be combined in water (e.g., purified water) with stirring, followed by pH adjustment to a suitable final pH. Techniques for preparing compositions may generally be found in “Remington's Pharmaceutical Sciences”, (Meade Publishing Co., Easton, Pa.).

When preparing compositions, components should be selected to optimize solubility, stability and compatibility. Compositions should typically be sterile and stable under the conditions of manufacture and storage. Compositions may be sterilized by filtering the composition through a sterilizing grade filter, such as a filter with a 0.22 micron nominal pore size.

### Methods of Evaluating Compositions

Compositions may be evaluated for stability using established procedures. For example, compositions may be subjected to accelerated stability testing. For example, compositions remain stable, and do not undergo precipitation or become cloudy when they are stored at 40° C. for at least 1 month, 3 months or 6 months prior to evaluation. The active component (e.g., a 6- or 7-aminoisoquinoline compound) should not react with other formulation components, or decompose. Methods of evaluating such compounds include, for example, high performance liquid chromatography (HPLC) or determination of optical rotation (e.g., to determine if a compound has racemized).

Compositions may also be evaluated using the Preservative Effectiveness Test of the United States Pharmacopoeia for parenteral/ophthalmic products. In such tests, which will be known to those skilled in the art, five indicator organisms are utilized for the purpose of challenging the preservative system in a product. Three of the five USP indicator organisms address the growth of bacteria: Escherichia coli, Pseudomonas aeruginosa, and Staphylococcus aureus. Candida albicans is the representative yeast, while Aspergillus niger is a mold. A product is inoculated (contaminated) with a number of organisms between 1×10⁵ (100,000) to 1×10⁶ (1,000,000) colony forming units (CFU) per mL of product. At various intervals, depending on the category, the composition is tested to determine its ability to control reproduction or destroy the microorganisms. A logarithmic reduction is evaluated at each test interval required for the category. By test definition, any growth over the allotted amount for any of the indicated microorganisms renders the preservative in the product not effective. Compositions may also be evaluated using the European Pharmacopoeia Preservative Effectiveness Test, which also evaluates growth of P. aeruginosa, S. aureus, C. albicans and A. niger. The compositions of the present disclosure will pass at least one of these preservative effectiveness tests.

### Methods of Use

One aspect of the disclosure relates to a method of treating disorder in a subject in need of treatment, comprising administering to the subject a safe and effective amount of a composition as described herein. In some embodiments, treatment can be for viral conjunctivitis and/or pulmonary manifestations of viral infection. In some embodiments, viral infection can be coronaviral infection. In some embodiments, viral infection can be severe acute respiratory syndrome coronavirus 2 (SARS-CoV-2).

The compositions can include one or more compounds as described herein such as, but not limited to an isoquinoline compound and/or a JAK inhibitor.

Another embodiment includes a method of treating an ocular disorder in a subject in need of treatment, comprising administering to the subject a safe and effective amount of a composition as described herein.

Another embodiment includes a method of treating viral conjunctivitis comprising administering to a subject in need thereof a safe and effective amount of a composition comprising a composition as described herein.

Routes of administration of any of the compositions described herein can include oral, nasal, inhaled, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful are not limited to the particular formulations and compositions that are described herein.

For oral application, particularly suitable are tablets, dragées, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

In embodiments, the compounds of the present disclosure may be topically administered. Topical compositions that can be applied locally to the eye may be in any form known in the art, non-limiting examples of which include drops, sprays, ointments, or a sustained or non-sustained release unit placed in the conjunctival cul-du-sac of the eye or another appropriate location.

In embodiments, the compounds may be administered by inhalation. Many methods of preparing inhaled formulations are known in the art, and any of them may be used.

Dosages may be varied based on the patient being treated, the condition being treated, the severity of the condition being treated, the route of administration, etc. to achieve the desired effect.

Administration of a compound or a composition described herein may result in a decrease in conjunctivitis, or an increase in pulmonary function, or the prevention of a decrease in pulmonary function, and a decrease in pulmonary fibrosis.

The following examples are intended to be illustrative, and should be considered to be non-limiting.

### Example 1

### Formulations

Topical pharmaceutical compositions for treating conjunctivitis are prepared by conventional methods and formulated as follows:

| | 1 | 2 | 3 | 4 |
|---|---|---|---|---|
| Formulation | (% w/w) | (% w/w) | (% w/w) | (% w/w) |
| netarsudil | 0.02 | 0.05 | 0.025 | 0.5 |
| axitinib | 0.04 | — | 0.4 | 0.004 |
| Boric acid | 0.05 | 0.05 | 0.05 | 0.05 |
| D-mannitol | 3.0 | 3.0 | 3.0 | 3.0 |
| Benzalkonium chloride | 0.015 | 0.015 | 0.015 | — |
| Polyoxyl 40 stearate | 0.5 | — | 0.5 | 0.5 |
| (Myrj-52) | | | | |
| Cremophor RH 40 | — | 0.5 | — | — |
| Polyethylene glycol | 2.5 | 2.5 | 2.5 | 2.5 |
| 400 (PEG-400) | | | | |
| EDTA | 0.01 | 0.01 | 0.01 | 0.01 |
| Purified water | q.s. | q.s. | q.s. | q.s. |

Formulations 1-3 were prepared by adding boric acid, D-mannitol, PEG-400, EDTA, and Myrj-52 or Cremophor RH40 in a labeled 150-milliliter (mL) plastic container. 100 milliliters (mL) of purified water were then added to bring the solution almost to 100%. The solution was stirred for 10 minutes. Stock solutions of 1.5% benzalkonium chloride, axitinib, netarsudil were then added and dissolved by stirring the solution for another 10 minutes, and the pH was adjusted to approximately 5.5.

Formulation 4 was prepared by adding boric acid, D-mannitol, PEG-400, EDTA, and Myrj-52 or Cremophor RH40 in a labeled 150-mL plastic container. 100 mL purified water was then added to bring the solution almost to 100%. The solution was stirred for 10 minutes. Axitinib and netarsudil were then added and dissolved by stirring the solution for another 10 minutes, and the pH was adjusted to approximately 5.5.

### Example 2

### Formulations

Topical pharmaceutical compositions for lowering intraocular pressure were prepared by conventional methods and formulated as follows:

| | 5 | 6 |
|---|---|---|
| Formulation | (% w/w) | (% w/w) |
| (1R,2R)-N-(4-methylisoquinolin-6-yl)- | 0.5 | 0.7 |
| 2-(4-(N-(pyridin-2- | | |
| yl)sulfamoyl)phenyl)cyclopropane-1- | | |
| carboxamide | | |
| ruxolitinib | 0.005 | 0.000 |
| Sodium Phosphate Monobasic | 0.031 | 0.0155 |
| Sodium Phosphate Dibasic | 0.07 | 0.0035 |
| Benzalkonium chloride | 0.015 | 0.015 |
| sodium chloride | 0.7 | 0.7 |
| EDTA | 0.05 | 0.05 |
| Purified water | q.s. | q.s. |

Formulations 5 and 6 were prepared by adding sodium phosphate monobasic, sodium phosphate dibasic, sodium chloride, and EDTA in a labeled 150-milliliter (mL) plastic storage container. 100 milliliter (mL) of purified water was then added to bring the solution almost to 100%. The solution was stirred for 10 minutes. Stock solutions of 1.5% benzalkonium chloride, and (1R,2R)—N-(4-methylisoquinolin-6-yl)-2-(4-(N-(pyridin-2-yl)sulfamoyl)phenyl)cyclopropane-1-carboxamide and ruxolitinib were then added and dissolved by stirring the solution for another 10 minutes, and the pH was adjusted to approximately 5.5.

### Example 3

### Formulations for Inhalation

Topical pharmaceutical compositions for inhalation were prepared by conventional methods and formulated as follows: 1 p.p.w. of netarsudil is added to a mixture comprising 0.65 p.p.w. Span 85, 21.22 p.p.w. Frigen 113 and 21.13 p.p.w. Frigen 11/12. The netarsudil dissolves in the Frigen components to provide a solution which is filled into the reservoir of a conventional inhaler device capable of delivering e.g., 1.0, 2.0, 5.0 or 10.0 mg at each actuation.

### Example 4

### Exemplary Combination Treatment

Formosan Rock macaque monkeys (Macaca cyclopis), animal identification consisting of uniquely numbered tattoos and color-coded cage cards are used in this study. On Study Day 1, the animals are at least four years old, and weigh at least 4 kg. The ocular tolerability is determined using a paired study design in which composition was administered q.d. AM for three days to one eye of each monkey (n=6 per group) with the untreated contralateral eye serving as an internal control. Each dose was administered just after the t=0, t=24, and t=48 hour measurement of intraocular pressure (IOP). IOP was taken in both eyes at time points of 0, 4, 8, 24, 48, 52, 56, and 72 hours after baseline (t=0) IOP measurement. Mortality observations, clinical observations, ocular irritation, and intraocular pressures were monitored, recorded, or measured throughout the in-life portion of the study. All treatments were administered as eye drops (one drop per eye). Each animal was sedated intramuscularly (IM) with approximately 5 mg/kg ketamine HCl (or to effect) with the objective of using the minimal dose necessary to achieve acceptable sedation to perform the IOP measurement and dosing procedure. A Model 30 CLASSIC pneumotonometer was used to measure intraocular pressure (IOP) non-invasively (Reichert, Inc, Depew, NY). One drop of ocular anaesthetic (0.5% proparacaine) was topically applied to each eye and allowed to take effect for at least 30 seconds prior to each IOP measurement. Using the pneumotonometer manual tonometry mode, and with the animal maintained in an upright position, 3 independent measurements were obtained and averaged for each eye, at all time points.

Three Rho Kinase inhibitor (ROCKi) formulations are prepared using the above Formulation one. When tested according to the above protocol, tolerability is demonstrated.

### Example 5

### Combination Treatment in Humans

A sterile, isotonic, aqueous solution is prepared as described above in Formula 4

Using the formulation above, a human diagnosed with CORVID-19 is treated twice daily with approximately 35 microliter drop(s) in both eyes for up to 7 days. Following this dosing regimen, a measurement of conjunctivitis shows a significant reduction from baseline.

### Example 6

### Combination Treatment in Humans

An inhaled formulation is prepared according to the above protocol.

Using the formulation above, a human diagnosed with pulmonary symptoms of CORVID-19 is treated twice daily. Following this dosing regimen, the patient maintains or increases lung function.

While the invention has been particularly shown and described with reference to particular embodiments, it will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method of reducing viral load comprising:
administering to a subject in need thereof an effective amount of a composition including a compound of Formula 1: 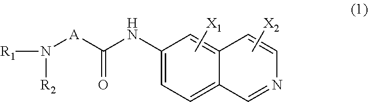
or a pharmaceutically acceptable salt thereof, wherein:
R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;
A is selected from the group consisting of —CH₂NH—, —CH(R₁₀)—, —C(CH₃)(R₁₀)—, —CH₂CH₂—, —CH(R₁₀)CH₂—, —CH₂CH₂CH(R₁₀)—, —CH₂CH(R₁₀)—, and —C(CH₃)(R₁₀)CH₂—;
each R₁₀ is independently selected from the group consisting of alkyl, alkenyl, alkynyl, amino, aryl, heteroaryl, cycloalkyl or heterocycloalkyl, any of which may be optionally substituted; and
X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl.

2. A method of reducing viral load comprising:
administering to a subject in need thereof an effective amount of a composition including a compound of Formula 5: 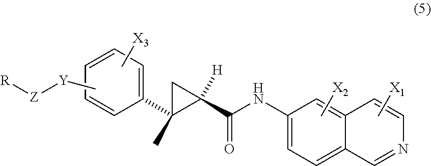
or tautomers, stereoisomers and pharmaceutically acceptable salts thereof,
wherein:
X₁, X₂ and X₃ are independently H, halogen, nitrile, hydroxyl, or C₁₋₆ alkyl;
Y is —NR^{N1}S(O)₂—, —NR^{N1}C(O)—, —O(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;
Z is a direct bond or C₁₋₆ alkylene;
R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;
R^{N1} is H or C₁₋₆ alkyl or forms a ring with R;
R¹ is H, F, or Me or forms a ring with R;
R³ is H or C₁₋₆ alkyl;
n is an integer from 0 to 6; and
m is an integer from 1 to 6.

3. The method of claim 1 or 2, wherein the viral load is a result of a coronavirus.

4. A method of treating viral conjunctivitis comprising:
topically administering to a subject in need thereof an effective amount of a composition including a compound of Formula 1: 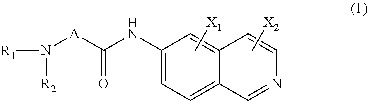
or a pharmaceutically acceptable salt thereof, wherein:
R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;
A is selected from the group consisting of —CH₂NH—, —CH(R₁₀)—, —C(CH₃)(R₁₀)—, —CH₂CH₂—CH(R₁₀)CH₂—, —CH₂CH₂CH(R₁₀)—, —CH₂CH(R₁₀)—, and —C(CH₃)(R₁₀)CH₂—;
each R₁₀ is independently selected from the group consisting of alkyl, alkenyl, alkynyl, amino, aryl, heteroaryl, cycloalkyl or heterocycloalkyl, any of which may be optionally substituted; and
X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl.

5. A method of treating viral conjunctivitis comprising:
topically administering to a subject in need thereof an effective amount of a composition including a compound of Formula 5: 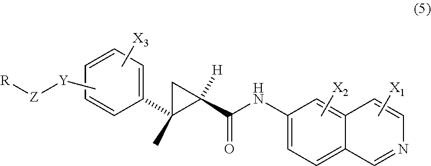
or tautomers, stereoisomers and pharmaceutically acceptable salts thereof,
wherein:
X₁, X₂ and X₃ are independently H, halogen, nitrile, hydroxyl, or C₁₋₆ alkyl;
Y is —NR^{N1}S(O)₂—, —NR^{N1}C(O)—, —O(CR¹₂)ₙ—, —NR^{N1}(CR¹₂)ₘ—, —C(O)O—, (O), —S(O)₂—, —C(O)—, —NR^{N1}C(O)O—, C₁₋₆ alkylene, aryl, or heteroaryl;
Z is a direct bond or C₁₋₆ alkylene;
R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;
R^{N1} is H or C₁₋₆ alkyl or forms a ring with R;
R¹ is H, F, or Me or forms a ring with R;
R³ is H or C₁₋₆ alkyl;
n is an integer from 0 to 6; and
m is an integer from 1 to 6.

6. The method of claim 4 or 5, wherein the viral conjunctivitis is a result a coronavirus.

7. A method of treating pulmonary manifestations of a viral infection comprising:
administering to a subject in need thereof an effective amount of a composition including a compound of Formula 1: 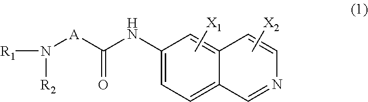
or a pharmaceutically acceptable salt thereof, wherein:
R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;
A is selected from the group consisting of —CH₂NH—, —CH(R₁₀)—, —C(CH₃)(R₁₀)—, —CH₂CH₂—CH(R₁₀)CH₂—, —CH₂CH₂CH(R₁₀)—, —CH₂CH(R₁₀)—, and —C(CH₃)(R₁₀)CH₂—;
each R₁₀ is independently selected from the group consisting of alkyl, alkenyl, alkynyl, amino, aryl, heteroaryl, cycloalkyl or heterocycloalkyl, any of which may be optionally substituted; and
X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl.

8. A method of treating pulmonary manifestations of a viral infection comprising:
administering to a subject in need thereof an effective amount of a composition including a compound of Formula 5: 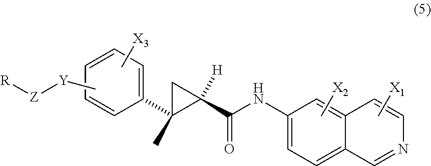
or tautomers, stereoisomers and pharmaceutically acceptable salts thereof,
wherein:
X₁, X₂ and X₃ are independently H, halogen, nitrile, hydroxyl, or C₁₋₆ alkyl;
Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;
Z is a direct bond or C₁₋₆ alkylene;
R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;
R^{N1} is H or C₁₋₆ alkyl or forms a ring with R;
R¹ is H, F, or Me or forms a ring with R;
R³ is H or C₁₋₆ alkyl;
n is an integer from 0 to 6; and
m is an integer from 1 to 6.

9. The method of claim 7 or 8, wherein the virus is a coronavirus.

10. The method of claim 7 or 8, wherein the administering is via inhalation.

11. The method of any preceding claim, further comprising administering at least one antiviral compound.

12. The method of any preceding claim, wherein the pulmonary manifestations are swelling, inflammation, fibrosis, or a combination thereof.

13. The method of any preceding claim, further comprising administering a ROCK/JAK inhibitor or a JAK inhibitor.

14. The method of claim 13, wherein the JAK inhibitor is tofacitinib, decernotinib, INCB18424, baricitinib, CYT387, GLPG0634, AC-430, axitinib, ruxolitinib, fibotinib, or peficitinib.

15. A composition comprising:
a compound of Formula 1: 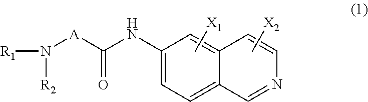
or a pharmaceutically acceptable salt thereof, wherein:
R₁ and R₂ are independently selected from the group consisting of hydrogen and C₁-C₄ alkyl, or R₁ and R₂ are taken together with the nitrogen atom to which they are attached to form a ring of 3, 4, 5, 6, 7 or 8 member atoms;
A is selected from the group consisting of —CH₂NH—, —CH(R₁₀)—, —C(CH₃)(R₁₀)—, —CH₂CH₂—CH(R₁₀)CH₂—, —CH₂CH₂CH(R₁₀)—, —CH₂CH(R₁₀)—, and —C(CH₃)(R₁₀)CH₂—;
each R₁₀ is independently selected from the group consisting of alkyl, alkenyl, alkynyl, amino, aryl, heteroaryl, cycloalkyl or heterocycloalkyl, any of which may be optionally substituted; and
X₁ and X₂ are independently selected from the group consisting of hydrogen, hydroxy, halogen, alkyl, amino, nitro, cyano, carbonyl, carbonylamino, alkoxy, aryloxy, sulfonyl, sulfonamido, thioalkyl, and carboxyl; and
at least one JAK inhibitor.

16. A composition comprising:
a compound of Formula 5: 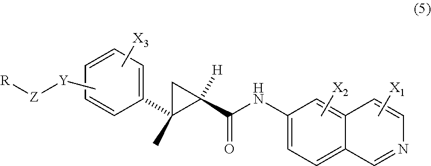
or tautomers, stereoisomers and pharmaceutically acceptable salts thereof,
wherein:
X₁, X₂ and X₃ are independently H, halogen, nitrile, hydroxyl, or C₁₋₆ alkyl;
Y is —NR^{N1}S(O)₂—, —NR^{N1}C(O)—, —(CR¹₂)ₙ—, —NR^{N1}(CR¹₂)ₘ—, —C(O)O—, —OC(O)—, —S(O)₂—, —C(O)—, —NR^{N1}C(O)O—, C₁₋₆ alkylene, aryl, or heteroaryl;
Z is a direct bond or C₁₋₆ alkylene;
R is H, halogen, cyano, OR³, C₁₋₆ alkyl, acyl, amino, cycloalkyl, heterocyclyl, aryl, or heteroaryl, or R may form a ring of 5 to 7 member atoms with R^{N1} or R¹, wherein the ring may contain up to 2 heteroatoms selected from N, O, and S, the ring being either saturated or unsaturated;
R^{N1} is H or C₁₋₆ alkyl or forms a ring with R;
R¹ is H, F, or Me or forms a ring with R;
R³ is H or C₁₋₆ alkyl;
n is an integer from 0 to 6; and
m is an integer from 1 to 6; and
at least one JAK inhibitor.

17. The composition of claim 15 or 16, wherein the composition is prepared as a powder for inhalation.

18. The composition of claim 15 or 16, wherein the composition is prepared for topical administration.

19. A method or composition as described herein.
